SEQUENCE LISTING (56 sequences)

SEQ ID NO: 1 - DNA, 19 bp, synthetic construct
  source 1..19: mol_type = other DNA
cgactacatccgcttcttg

SEQ ID NO: 2 - DNA, 20 bp, synthetic construct
  source 1..20: mol_type = other DNA
ggtccttcagtgatttgctt

SEQ ID NO: 3 - DNA, 21 bp, synthetic construct
  source 1..21: mol_type = other DNA
aatgctgctcttgatgctctc

SEQ ID NO: 4 - DNA, 21 bp, synthetic construct
  source 1..21: mol_type = other DNA
aaagagactgaaccgaaggca

SEQ ID NO: 5 - DNA, 18 bp, synthetic construct
  source 1..18: mol_type = other DNA
gcgaccaggaagaagaga

SEQ ID NO: 6 - DNA, 19 bp, synthetic construct
  source 1..19: mol_type = other DNA
acaaatcccacagagtcca

SEQ ID NO: 7 - DNA, 20 bp, synthetic construct
  source 1..20: mol_type = other DNA
gtaagcgcagttccttccgc

SEQ ID NO: 8 - DNA, 21 bp, synthetic construct
  source 1..21: mol_type = other DNA
cggatctcaatggaggccaag

SEQ ID NO: 9 - DNA, 20 bp, synthetic construct
  source 1..20: mol_type = other DNA
ctcaactgtgaaatgccacc

SEQ ID NO: 10 - DNA, 20 bp, synthetic construct
  source 1..20: mol_type = other DNA
cttcatcttttggggtccgt

SEQ ID NO: 11 - DNA, 20 bp, synthetic construct
  source 1..20: mol_type = other DNA
agacggcttgtgccatcatt

SEQ ID NO: 12 - DNA, 20 bp, synthetic construct
  source 1..20: mol_type = other DNA
ctgcctccactagcatccag

SEQ ID NO: 13 - DNA, 21 bp, synthetic construct
  source 1..21: mol_type = other DNA
gctacaagaggatcaccagca

SEQ ID NO: 14 - DNA, 21 bp, synthetic construct
  source 1..21: mol_type = other DNA
gcacagacctctctcttgagc

SEQ ID NO: 15 - DNA, 21 bp, synthetic construct
  source 1..21: mol_type = other DNA
tgtctactcccaggttctctt

SEQ ID NO: 16 - DNA, 19 bp, synthetic construct
  source 1..19: mol_type = other DNA
atagcaaatcggctgacgg

SEQ ID NO: 17 - DNA, 18 bp, synthetic construct
  source 1..18: mol_type = other DNA
cctttcctcctccctctg

SEQ ID NO: 18 - DNA, 22 bp, synthetic construct
  source 1..22: mol_type = other DNA
tgcctctactgaatgattacac

SEQ ID NO: 19 - DNA, 18 bp, synthetic construct
  source 1..18: mol_type = other DNA
caggaggcagacactgat

SEQ ID NO: 20 - DNA, 18 bp, synthetic construct
  source 1..18: mol_type = other DNA
cggattaagcagcagcaa

SEQ ID NO: 21 - DNA, 18 bp, synthetic construct
  source 1..18: mol_type = other DNA
cctcagcctccatcttct

SEQ ID NO: 22 - DNA, 20 bp, synthetic construct
  source 1..20: mol_type = other DNA
gaccttacaaccaagcaact

SEQ ID NO: 23 - DNA, 22 bp, synthetic construct
  source 1..22: mol_type = other DNA
cggacctctaagatctaactac

SEQ ID NO: 24 - DNA, 20 bp, synthetic construct
  source 1..20: mol_type = other DNA
ctacctttcccattcccttc

SEQ ID NO: 25 - DNA, 20 bp, synthetic construct
  source 1..20: mol_type = other DNA
gtgaaggtcagaatgcaagc

SEQ ID NO: 26 - DNA, 20 bp, synthetic construct
  source 1..20: mol_type = other DNA
agggcccccttcatgaggtc

SEQ ID NO: 27 - DNA, 21 bp, synthetic construct
  source 1..21: mol_type = other DNA
acttgcctcactactgtcctt

SEQ ID NO: 28 - DNA, 19 bp, synthetic construct
  source 1..19: mol_type = other DNA
tgctggtatcggctcaata

SEQ ID NO: 29 - DNA, 20 bp, synthetic construct
  source 1..20: mol_type = other DNA
ctggatgtttgcagagcacg

SEQ ID NO: 30 - DNA, 20 bp, synthetic construct
  source 1..20: mol_type = other DNA
tcgacccgagaagaccttga

SEQ ID NO: 31 - DNA, 22 bp, synthetic construct
  source 1..22: mol_type = other DNA
tggagtgacatagagtgtgctg

SEQ ID NO: 32 - DNA, 24 bp, synthetic construct
  source 1..24: mol_type = other DNA
tcagaaaggtcaagttcaggaaga

SEQ ID NO: 33 - DNA, 24 bp, synthetic construct
  source 1..24: mol_type = other DNA
acactaacatatcaacaagaggag

SEQ ID NO: 34 - DNA, 18 bp, synthetic construct
  source 1..18: mol_type = other DNA
cattgccaggaagaccag

SEQ ID NO: 35 - DNA, 19 bp, synthetic construct
  source 1..19: mol_type = other DNA
tagacgaagccacgaagta

SEQ ID NO: 36 - DNA, 18 bp, synthetic construct
  source 1..18: mol_type = other DNA
gagcctagcgagttcaac

SEQ ID NO: 37 - DNA, 20 bp, synthetic construct
  source 1..20: mol_type = other DNA
gccctcctttcgtgtgagtt

SEQ ID NO: 38 - DNA, 20 bp, synthetic construct
  source 1..20: mol_type = other DNA
cacaggctcccgactaatgg

SEQ ID NO: 39 - DNA, 20 bp, synthetic construct
  source 1..20: mol_type = other DNA
attgctgtgtggggcagatt

SEQ ID NO: 40 - DNA, 20 bp, synthetic construct
  source 1..20: mol_type = other DNA
ccacgatttgcgtgatgtcc

SEQ ID NO: 41 - DNA, 20 bp, synthetic construct
  source 1..20: mol_type = other DNA
ccctggactttgagggcaat

SEQ ID NO: 42 - DNA, 20 bp, synthetic construct
  source 1..20: mol_type = other DNA
ccaagcctgtatcttgcacc

SEQ ID NO: 43 - DNA, 20 bp, synthetic construct
  source 1..20: mol_type = other DNA
aagagactgctggcataggg

SEQ ID NO: 44 - DNA, 20 bp, synthetic construct
  source 1..20: mol_type = other DNA
aagagactgctggcataggg

SEQ ID NO: 45 - DNA, 21 bp, synthetic construct
  source 1..21: mol_type = other DNA
ggtgtgatttccaaagccagt

SEQ ID NO: 46 - DNA, 20 bp, synthetic construct
  source 1..20: mol_type = other DNA
gtgtcgtcttccctccgttt

SEQ ID NO: 47 - DNA, 20 bp, synthetic construct
  source 1..20: mol_type = other DNA
ggccccaactttgagactgt

SEQ ID NO: 48 - DNA, 20 bp, synthetic construct
  source 1..20: mol_type = other DNA
gggactgtgctcatgccaac

SEQ ID NO: 49 - DNA, 21 bp, synthetic construct
  source 1..21: mol_type = other DNA
atgctcaaggtgatagctcgg

SEQ ID NO: 50 - DNA, 20 bp, synthetic construct
  source 1..20: mol_type = other DNA
tccagaaccgagaactgctg

SEQ ID NO: 51 - DNA, 21 bp, synthetic construct
  source 1..21: mol_type = other DNA
aactcaactgccaagtgtcct

SEQ ID NO: 52 - DNA, 20 bp, synthetic construct
  source 1..20: mol_type = other DNA
cttgacctctgccaccttga

SEQ ID NO: 53 - DNA, 20 bp, synthetic construct
  source 1..20: mol_type = other DNA
cgaaggtttgtgggtttggg

SEQ ID NO: 54 - DNA, 21 bp, synthetic construct
  source 1..21: mol_type = other DNA
gccttaacacaacgctccatc

SEQ ID NO: 55 - DNA, 20 bp, synthetic construct
  source 1..20: mol_type = other DNA
agccaatcagcgttcggtat

SEQ ID NO: 56 - DNA, 20 bp, synthetic construct
  source 1..20: mol_type = other DNA
ggccagtacaatcagggctc

(19) United States
(12) Patent Application Publication
(10) Pub. No.: US 2025/0161301 A1
(43) Pub. Date: May 22, 2025

(54) COMPOSITION FOR PREVENTING, AMELIORATING OR TREATING OBESITY AND DIABETES COMPRISING ZINC GLUCONATE AND CYCLO-HISPRO AS ACTIVE INGREDIENTS

(71) Applicant: NOVMETAPHARMA CO., LTD, Seoul (KR)

(72) Inventors: Hoe Yune JUNG, Pohang-si (KR); Jongsu JEON, Pohang-si (KR); Do Hyun LEE, Pohang-si (KR); Seo Yeong BAEK, Pohang-si (KR); Shin Yu BAEK, Pohang-si (KR); Heon Jong LEE, Incheon (KR)

(73) Assignee: NOVMETAPHARMA CO., LTD, Seoul (KR)

(21) Appl. No.: 18/954,719

(22) Filed: Nov. 21, 2024

(30) Foreign Application Priority Data
Nov. 21, 2023 (KR) 10-2023-0162749
Apr. 26, 2024 (JP) 2024-073020
Apr. 26, 2024 (KR) 10-2024-0056071

(51) Int. Cl. A61K 31/4985; A61K 33/30; A61P 3/04; A61P 3/10

(52) U.S. Cl. CPC A61K 31/4985; A61K 33/30; A61P 3/04; A61P 3/10

(57) ABSTRACT

A composition for preventing, ameliorating or treating obesity and diabetes and its uses are disclosed. The composition includes zinc gluconate and cyclo-hispro (CHP) as active ingredients. More specifically, the composition includes the type of zinc salts and/or the content ratio of zinc components optimized for surprisingly significantly improved anti-obesity and anti-diabetic effects. A method of preventing, ameliorating or treating obesity and diabetes using the composition is also disclosed.

### CROSS-REFERENCE TO RELATED APPLICATION

This application claims priority to and the benefit of Korean Patent Application No. 10-2023-0162749, filed on Nov. 21, 2023, Korean Patent Application No. 10-2024-0056071, filed on Apr. 26, 2024 and Japanese Patent Application No. 2024-073020, filed on Apr. 26, 2024, the disclosures of which are incorporated herein by reference in their entireties.

### INCORPORATION BY REFERENCE OF SEQUENCE LISTING

The content of the electronically submitted sequence listing, file name: Q297851_Sequence_listing.xml; size: 49.2 KB; and date of creation: Nov. 20, 2024, filed herewith, is incorporated herein by reference in its entirety.

### BACKGROUND

### 1. Field of the Invention

The present invention relates to a composition for preventing, ameliorating or treating obesity and diabetes, which includes zinc gluconate and cyclo-hispro (CHP) as active ingredients, and more particularly, to a composition in which the type of zinc salts and/or the content ratio of zinc components is optimized to significantly improve anti-obesity and anti-diabetic effects, and a method of preventing, ameliorating or treating obesity and diabetes using the same.

### 2. Discussion of Related Art

Type 2 diabetes mellitus (T2DM) is a metabolic disorder characterized by hyperglycemia and insulin resistance in various organs. Healthy lifestyles, including exercise, proper diet, and weight control, can help manage the disease. However, as the disease progresses, oral drug treatment or insulin therapy is often required. Currently available oral drugs such as hypoglycemic agents or insulin sensitizers, including sulfonylurea (SU), biguanides, thiazolidinedione (TZD), dipeptidyl peptidase-4 (DPP-4) inhibitors, and sodium-glucose cotransporter 2 (SGLT2) inhibitors, have been used to regulate blood glucose levels for a long time. However, some drugs have limited efficacy and may cause various side effects. For example, patients taking SU have increased risks of weight gain and hypoglycemia, and patients taking biguanides are at potential risk of lactic acidosis. TZD is not recommended for patients with existing edema, heart failure, or acute liver disease. The most common adverse reaction of DPP-4 inhibitors is upper respiratory tract infection, while SGLT2 inhibitors are associated with urinary tract and genital tract infections. Therefore, there is a need to develop safer and more effective drugs with various mechanisms of action.

Lysine acetylation plays an important role in maintaining energy homeostasis in various metabolic pathways. In particular, several enzymes involved in glucose and lipid metabolism are regulated by the acetylation of lysine residues. The sirtuin family of enzymes consists of increased levels of β-nicotinamide adenine dinucleotide (NAD⁺)-dependent deacetylases that regulate the activities of many other enzymes. Peroxisome proliferator-activated receptor gamma coactivator 1-alpha (PGC-1α), which is a positive regulator of mitochondrial biogenesis, is regulated by sirtuin 1 (Sirt1). Sirt1 also regulates the acetylation of liver kinase B1 (LKB1), a major AMP-activated kinase (AMPK) involved in lipid synthesis and fatty acid oxidation. Sirtuin has also been considered as a novel target for the treatment of chronic metabolic diseases, and enhancement of Sirt1 activity has been reported to reverse the pathological effects of T2DM.

Meanwhile, Patent Document 1 discloses a composition including zinc ions and cyclo-hispro (CHP) as a composition useful for attenuating the symptoms of diabetes in mammals, but nothing is known about the optimized form of a zinc salt to improve the therapeutic effects on obesity and diabetes.

Based on this background, the present inventors have found that a combination of zinc gluconate and CHP exhibits significantly superior anti-obesity and anti-diabetic effects compared to other combinations of zinc salts and CHP. Therefore, the present invention has been completed based on this finding.

### RELATED-ART DOCUMENT

### Patent Document

Patent Document 1: Korean Patent Publication No. 10-2001-0022786

### SUMMARY OF THE INVENTION

Accordingly, the present invention is directed to providing a composition for preventing, ameliorating or treating obesity and diabetes, which includes zinc gluconate and cyclo-hispro or a pharmaceutically acceptable salt thereof as active ingredients.

The present invention is also directed to providing a method of preventing, ameliorating or treating obesity and diabetes in a subject in need thereof, which includes: administering to the subject an effective amount of a composition comprising zinc gluconate and cyclo-hispro or a pharmaceutically acceptable salt thereof.

In order to solve the above problems, according to an aspect of the present invention, there is provided a pharmaceutical composition for preventing or treating obesity and diabetes, which includes zinc gluconate and cyclo-hispro or a pharmaceutically acceptable salt thereof as active ingredients.

According to another aspect of the present invention, there is provided a health functional food composition preventing or ameliorating obesity and diabetes, which includes zinc gluconate and cyclo-hispro or a pharmaceutically acceptable salt thereof as active ingredients.

According to still another aspect of the present invention, there is provided a method of preventing, ameliorating or treating obesity and diabetes in a subject in need thereof, which includes: administering to the subject an effective amount of a composition comprising zinc gluconate and cyclo-hispro or a pharmaceutically acceptable salt thereof.

In the present invention, the zinc cation or zinc element component of the zinc gluconate and cyclo-hispro or a pharmaceutically or sitologically acceptable salt thereof may be included or administered in a weight ratio of 1 to 5:1 to 4.

In the present invention, the zinc gluconate and cyclo-hispro or a pharmaceutically or sitologically acceptable salt thereof may be included or administered in a dose of 15 to 250 mg.

In the present invention, the zinc gluconate and cyclo-hispro or a pharmaceutically or sitologically acceptable salt thereof may be included or administered in a weight ratio of 7 to 35:1 to 4.

In the present invention, the diabetes may be type 2 diabetes accompanied by obesity.

In the present invention, the composition may exhibit anti-obesity and anti-diabetic effects through an increase in NAD⁺ synthesis, which regulates the Sirt1 deacetylase activity in liver and visceral adipose tissues.

### BRIEF DESCRIPTION OF THE DRAWINGS

FIG. 1 shows the weight loss effect of administration of CycloZ, which includes various forms of zinc salts, in KKAy mice that are a model of diabetes and obesity.

FIG. 2 shows the effect of lowering blood glycated hemoglobin (HbA1c) with administration of CycloZ, which includes various forms of zinc salts, in KKAy mice that are a model of diabetes and obesity.

FIGS. 3A and 3B show (A) the change in fasting blood glucose and (B) the area under the curve of blood glucose levels during an oral glucose tolerance test for 2 hours with administration of CycloZ, which includes various forms of zinc salts, in KKAy mice that are a model of diabetes and obesity, respectively.

FIGS. 4A and 4B show the results of confirming (A) the change in fasting blood glucose and (B) the change in blood glycated hemoglobin (HbA1c) with individual or combined administration of zinc gluconate and CHP in KKAy mice that are a model of diabetes and obesity.

FIGS. 4C to 4F show the results of confirming (C) the change in calorie intake, (D) the change in blood low-density lipoprotein cholesterol level, (E) the change in total blood cholesterol level, and (F) the frequency of adipocyte size in white adipose tissue, in that order, with the combined administration of zinc gluconate and CHP in KKAy mice that are a model of diabetes and obesity, respectively (^{a}P≤0.05, ^{b}P≤0.01, ^{c}P≤0.001).

FIG. 5A to FIG. 5M show (A) the changes in body weights, (B) the changes in weights of respective organs, (C) the oral glucose tolerance test results, (D) the change in fasting blood glucose, (E) the change in plasma glycated hemoglobin (HbA1c), (F) the change in plasma insulin concentration, (G) the change in plasma free fatty acid concentration, (H) the change in plasma triglyceride concentration, (I) the change in plasma high-density lipoprotein concentration, (J) the change in plasma adiponectin level, (K) the changes in mRNA expression levels of genes involved in fatty acid and cholesterol synthesis in the liver, (L) the hematoxylin and eosin staining results in the liver, and (M) the hematoxylin and eosin staining results in the epididymal adipose tissue (EAT), in that order, with the combined administration of zinc gluconate and CHP in KKAy mice that are a model of diabetes and obesity, respectively (^{a}P≤0.05, ^{b}P≤0.01, ^{c}P≤0.001, ^{d}P≤0.0001).

FIGS. 6A to 6E show (A) the changes in mRNA expression levels of genes related to inflammatory cytokines and infiltrated monocytes in the liver and mesenteric adipose tissue, (B) the changes in TNFα and MCP-1 protein levels in the liver, (C) the changes in TNFα and MCP-1 protein levels in EAT, (D) the change in expression of TNFα, MCP-1, F4/80, and CD11b in the liver measured by immunohistochemistry, and (E) the change in expression of TNFα, MCP-1, F4/80, and CD11b in EAT measured by immunohistochemistry, in that order, with the combined administration of zinc gluconate and CHP in KKAy mice that are a model of diabetes and obesity, respectively (^{a}P≤0.05, ^{b}P≤0.01, ^{c}P≤0.0001).

FIGS. 7A to 7H show (A) the change in acetylated lysine level of p65 in the liver and white adipose tissue (WAT), (B) the changes in acetylated lysine levels for PGC-1α in the liver and EAT, (C) the changes in acetylated lysine levels for LKB1 in the liver and EAT, (D) phosphorylated AMPK (T172) in the liver and EAT, (E) the changes in mRNA expression levels of genes related to the mitochondrial biogenesis in the liver and MAT, (F) the changes in mRNA expression levels of genes related to the lipid oxidation in the liver and MAT, (G) the results of measuring oxygen consumption rates in AML12 with and without palmitate, and (H) the mitochondrial mass measured by MitoTracker staining, in that order, with the combined administration of zinc gluconate and CHP in KKAy mice that are a model of diabetes and obesity, respectively (^{a}P≤0.05, ^{b}P≤0.01, ^{c}P≤0.001, ^{d}P≤0.0001).

FIGS. 8A to 8E show (A) the global acetyl-lysine level in the liver tissue, (B) mitochondrial DNA (mtDNA) in the liver measured by mitochondrial D-loop PCR, (C) increased levels of NAD+/NADH ratio in skeletal muscle, (D) the Sirt1 protein expression in the liver and EAT, and (E) the change in Sirt1 mRNA expression level in the liver and EAT, in that order, with the combined administration of zinc gluconate and CHP in KKAy mice that are a model of diabetes and obesity, respectively (^{a}P≤0.05, ^{b}P≤0.01).

FIGS. 9A to 9D show (A) the NAD⁺/NADH ratio in the liver and EAT, (B) the NAD⁺ quantification results in the liver and EAT, (C) the changes in mRNA expression levels of genes related to the NAD⁺ synthesis in the liver, and (D) the changes in mRNA expression levels of genes related to the NAD⁺ synthesis in MAT, in that order, with the combined administration of zinc gluconate and CHP in KKAy mice that are a model of diabetes and obesity, respectively (^{a}P≤0.05, ^{b}P≤0.01, ^{c}P≤0.001).

FIGS. 10A to 10G show (A) the change in glycated hemoglobin (HbA1c) in KKAy mice that are a model of diabetes and obesity, and (B) the changes in body weights, (C) the cumulative potassium intake, (D) the weights of respective organs, (E) the changes in mRNA expression levels of genes related to the mitochondrial biogenesis in the liver, (F) the changes in mRNA expression levels of genes related to the lipid oxidation in the liver, and (G) the global acetyl-lysine level in the liver, in that order, with the combined administration of zinc gluconate and CHP in KKAy mice that are a model of diabetes and obesity, respectively (^{a}P≤0.05, ^{b}P≤0.01, ^{c}P≤0.001, ^{d}P≤0.0001).

FIGS. 11A to 11F show (A) the oral glucose tolerance test results, (B) the change in glycated hemoglobin (HbA1c), (C) the acetylated lysine levels for PGC-1α and LKB1 in the liver, (D) phosphorylated AMPK (T172) in the liver, (E) the increased levels of NAD⁺/NADH ratio and results of NAD⁺ quantification results in the liver, and (F) the changes in mRNA expression levels of genes related to the NAD⁺ synthesis in the liver in KKAy mice that are a model of severe diabetes in that order, respectively (^{a}P≤0.05, ^{b}P≤0.01, ^{c}P≤0.001, ^{d}P≤0.0001).

### DETAILED DESCRIPTION OF EXEMPLARY EMBODIMENTS

Hereinafter, the present invention will be described in further detail.

Unless otherwise defined, all technical terms used in the present invention are used with the same meanings as generally understood by those skilled in the art to which the present invention pertains. Also, preferred methods or test samples are described in this specification, but those similar or equivalent to these methods are also included in the scope of the present invention.

As described above, nothing is known about the optimized form of a zinc salt to improve the therapeutic effects on obesity and diabetes when used in a combination of a zinc salt and CHP. Accordingly, the present inventors have sought a solution to the above-described problem by experimentally verifying that a combination of zinc gluconate and CHP shows superior obesity improvement and diabetes treatment effects compared to other combinations of zinc salts and CHP.

Therefore, a first aspect of the present invention relates to a pharmaceutical composition for preventing or treating obesity and diabetes, which includes zinc gluconate and cyclo-hispro or a pharmaceutically acceptable salt thereof as active ingredients.

As used herein, the term “cyclo-hispro (CHP)” refers to a naturally occurring cyclic dipeptide consisting of histidine and proline, which are metabolites of thyrotropin-releasing hormone (TRH), or a bioactive dipeptide that is also synthesized de novo in the body through TRH metabolic processes, and refers to a substance widely distributed throughout the brain and in the spinal cord, gastrointestinal tract, and the like.

In the composition of the present invention, the cyclo-hispro may be synthesized and used, or a commercially available product may be used. Also, the cyclo-hispro may be purified from cyclo-hispro-containing materials, for example, a prostate extract, a soybean hydrolysate, and the like, and used.

The term “purified” is used to mean that the cyclo-hispro is in a concentrated form compared to forms that can be obtained from natural sources such as a prostate extract. Purified ingredients may be concentrated from natural sources thereof, or obtained through chemical synthesis methods.

In this specification, the zinc salt and cyclo-hispro are also referred to as “CycloZ.” In this case, when the form of the zinc salt is zinc gluconate, it may be referred to as “gluconate-CycloZ.” Zinc gluconate and cyclo-hispro may be included in the composition of the present invention in the form of a single complex or as individual components. Accordingly, zinc gluconate and cyclo-hispro may be administered in the form of a single complex, or administered simultaneously, separately, or sequentially as individual components.

In the present invention, the zinc cation or zinc element component of the zinc gluconate and cyclo-hispro or a pharmaceutically acceptable salt thereof may be included in a weight ratio of 1 to 10:1 to 5, preferably a weight ratio of 1 to 5:1 to 4 or 1 to 5:1 to 3 or 1 to 5:1 to 2.

In the present invention, zinc gluconate and cyclo-hispro (gluconate-Cyclo-Z) may be included in a dose of 15 to 250 mg in the form of a single complex when applied clinically, and if zinc gluconate and cyclo-hispro are included as individual components, they may be included in a dose calculated according to the weight ratio of zinc gluconate: cyclo-hispro as defined in the present invention within the above dose range. If the total dose of zinc gluconate and cyclo-hispro (gluconate-Cyclo-Z) is included in less than 15 mg, the treatment effect for obesity and diabetes may not be achieved due to the low dose, and if the total dose is included in excess of 250 mg, toxicity problems may arise.

In the present invention, the zinc gluconate and cyclo-hispro or a pharmaceutically acceptable salt thereof may be included in a weight ratio of 7 to 35:1 to 4, preferably a weight ratio of 7 to 35:1 to 3 or 7 to 35:1 to 2. When the zinc gluconate and cyclo-hispro are included in contents falling out of the above weight ratio, the therapeutic effects on obesity and diabetes may be reduced.

In the present invention, the diabetes may be type 2 diabetes, or type 2 diabetes accompanied by obesity.

The composition of the present invention exhibits beneficial effects on both obesity and diabetes from a prophylactic and therapeutic standpoint. Young KK-Ay mice are used as a model of mild hyperglycemia for prophylactic treatment of type 2 diabetes accompanied by obesity. Hyperglycemia and hyperinsulinemia in KK-Ay mice worsen with age. Accordingly, in the present invention, the young KK-Ay mice and old KK-Ay mice with progressive hyperglycemia were used in separate studies to verify the therapeutic effect of gluconate-Cyclo-Z in a model of mild hyperglycemia and a model of severe diabetes.

According to one specific embodiment of the present invention, gluconate-CycloZ exhibits anti-obesity and anti-diabetic effects through regulation of protein acetylation in the liver and VAT. Increased VAT mass is one of the major risk factors for various metabolic diseases, and administration of gluconate-CycloZ significantly decreased liver and VAT mass in the KK-Ay mice. Also, PGC-1α deacetylation by administration of gluconate-CycloZ induced transcriptional regulation of genes related to the mitochondrial function in the liver and VAT. It is known that there is a close relationship between the PGC-1α activity and the onset of type 2 diabetes, which is related to the mitochondrial biogenesis and glucose/fatty acid metabolism. Specifically, reduced activity of PGC-1α is associated with altered lipid oxidation, and expression of PGC-1α in adipose tissue is down-regulated in patients with type 2 diabetes.

Chronic inflammation with abnormally increased cytokine levels and immune cell infiltration is observed in many metabolic disorders, and thus contributes to the progression of diseases. Therefore, in one specific embodiment of the present invention, a decrease in inflammation in the liver and VAT of KK-Ay mice treated with gluconate-CycloZ was observed. It was confirmed that acetylation of p65, which is a core NF-κB subunit, decreased when gluconate-CycloZ was administered.

According to another specific embodiment of the present invention, it was confirmed that gluconate-CycloZ regulates the expression of enzymes involved in the NAD⁺ synthesis. The mRNA expression of Nampt increased in both the liver and VAT of gluconate-CycloZ-treated mice. Because NAMPT is a rate-limiting enzyme, gluconate-CycloZ treatment increased an NAD⁺ level, which eventually increased Sirt1 activity. The NAD⁺/NADH ratio and the amount of NAD⁺ increased only in the liver and VAT, and did not increase in muscle. This indicates that gluconate-CycloZ may regulate the NAD⁺ synthesis in a tissue-specific manner.

According to still another specific embodiment of the present invention, it was confirmed that gluconate-CycloZ increases Sirt1 mRNA and protein expression in the liver and EAT of KK-Ay mice. In the present invention, it may be assumed that the Sirt1 activity may have increased because the NAD⁺ levels and NAD⁺/NADH ratio essential for Sirt1 activity increased.

In summary, this indicates that gluconate-CycloZ increases the NAD⁺ levels and reduces the protein acetylation by regulating the activity of NAD⁺-dependent deacetylases such as sirtuin.

As a result, gluconate-CycloZ activates the Sirt1/PGC-1α/LKB1/AMPK signaling axis, thereby exhibiting anti-obesity and anti-diabetic properties and an excellent safety profile. Based on these data, gluconate-CycloZ acts as a novel NAD⁺ booster and an Sirt1 deacetylase activator, the mechanisms of actions of which are different from those of existing type 2 diabetes drugs.

As used in the present invention, the term “prevention” refers to any action that suppresses or delays the onset of a disease or a condition. In the present invention, the prevention means delaying or suppressing the onset of obesity and diabetes.

As used in the present invention, the term “improvement” refers to any action that improves or beneficially changes a disease or a condition. In the present invention, the improvement means improving the symptoms of obesity and diabetes.

As used in the present invention, the term “treatment” refers to any action that delays, stops, or reverses the progression of a disease or a condition. In the present invention, the treatment means alleviating, relieving, eliminating, or reversing the symptoms of obesity and diabetes.

As used in the present invention, the term “pharmaceutically acceptable salt” refers to any organic or inorganic addition salt of cyclo-hispro that is preset at a concentration sufficient to exhibit an effective effect that is relatively non-toxic and harmless to patients, that is, any organic or inorganic addition salt in which the side effects caused by such an acceptable salt do not hinder the beneficial efficacy of cyclo-hispro. For these salts, inorganic acids and organic acids may be used as free acids. In this case, hydrochloric acid, hydrobromic acid, nitric acid, sulfuric acid, perchloric acid, phosphoric acid, and the like may be used as the inorganic acids, and citric acid, acetic acid, lactic acid, maleic acid, fumaric acid, gluconic acid, methanesulfonic acid, glyconic acid, succinic acid, tartaric acid, galacturonic acid, embonic acid, glutamic acid, aspartic acid, oxalic acid, (D) or (L) malic acid, maleic acid, methanesulfonic acid, ethanesulfonic acid, 4-toluenesulfonic acid, salicylic acid, citric acid, benzoic acid, malonic acid, or the like may be used as the organic acids. Also, these salts include alkali metal salts (sodium salts, potassium salts, and the like), alkaline earth metal salts (calcium salts, magnesium salts, and the like), and the like. For example, acid addition salts may include acetate, aspartate, benzoate, besylate, bicarbonate/carbonate, bisulfate/sulfate, borate, camsylate, citrate, edisylate, esylate, formate, fumarate, gluceptate, gluconate, glucuronate, hexafluorophosphate, hybenzate, hydrochloride/chloride, hydrobromide/bromide, hydroiodide/iodide, isethionate, lactate, malate, maleate, malonate, mesylate, methyl sulfate, naphthylate, 2-naphsylate, nicotinate, nitrate, orotate, oxalate, palmitate, pamoate, phosphate/hydrogen phosphate/dihydrogen phosphate, saccharate, stearate, succinate, tartrate, tosylate, trifluoroacetate, aluminum, arginine, benzathine, calcium, choline, diethylamine, diolamine, glycine, lysine, magnesium, meglumine, olamine, potassium, sodium, tromethamine, zinc salts, and the like.

The pharmaceutical composition of the present invention may further include a pharmaceutically acceptable carrier. The pharmaceutical composition including the pharmaceutically acceptable carrier may be prepared into various oral or parenteral formulations. When formulated, the pharmaceutical composition may be prepared using diluents or excipients such as commonly used fillers, extenders, binders, wetting agents, disintegrants, surfactants, and the like. Solid preparations for oral administration may include tablets, lozenges, powders, granules, capsules, troches, and the like. These solid preparations may be prepared by mixing one or more compounds of the present invention with at least one or more excipients, such as starch, calcium carbonate, sucrose, lactose, gelatin, or the like. Also, in addition to simple excipients, lubricants such as magnesium styrate talc may also be used. Suspensions, oral liquids, emulsions, syrups, or the like may be used as liquid preparations for oral administration. In addition to the commonly used simple diluents such as water and liquid paraffin, the liquid preparations for oral administration may include various excipients such as wetting agents, sweeteners, fragrances, preservatives, and the like.

Preparations for parenteral administration may include sterilized aqueous solutions, non-aqueous solutions, suspensions, emulsions, freeze-dried preparations, suppositories, and the like. Propylene glycol, polyethylene glycol, vegetable oils such as olive oil, injectable esters such as ethyl oleate, and the like may be used as non-aqueous solvents and suspending agents. Witepsol, Macrogol, Tween 61, cacao butter, laurin butter, glycerol, gelatin, and the like may be used as a base for suppositories.

The pharmaceutical composition of the present invention may be administered through any general route through which the pharmaceutical composition may reach the target tissue or cells in a subject or test sample. The administration may include systemic or local administration. Specifically, the administration may include intraperitoneal administration, intravenous administration, intramuscular administration, subcutaneous administration, intradermal administration, oral administration, intranasal administration, intrapulmonary administration, intrarectal administration, and the like, but the present invention is not limited thereto. The dose of the pharmaceutical composition may vary depending on the age, weight, and sex of a patient, the dosage form, the health condition, and the disease severity.

The term “patient” refers to any single subject in need of treatment, including humans, cattle, dogs, guinea pigs, rabbits, chickens, insects, and the like. Also, the subject may be any subject participating in a clinical research trial who does not show any clinical signs of disease, a subject participating in an epidemiological study, or a subject used as a control.

A second aspect of the present invention relates to a health functional food composition for preventing or ameliorating obesity and diabetes, which includes zinc gluconate, and cyclo-hispro or a sitologically acceptable salt thereof as active ingredients.

In the health functional food composition of the present invention, the description of configurations and effects of zinc gluconate and cyclo-hispro included as the active ingredients is the same as described above and is therefore omitted.

In the present invention, the term “sitologically acceptable salt” includes salts derived from sitologically acceptable organic acids, inorganic acids or bases.

In the present invention, the term “health functional food” encompasses all meanings of “functional food” and “health food.”

In the present invention, the term “functional food” is the same as the term “food for special health use (FoSHU)” and refers to a food with potent pharmaceutical and medical effects which are processed so that bio-regulatory functions can be efficiently exhibited.

In the present invention, the term “health food” refers to a food having an active health maintenance or enhancement effect compared to general foods, and a health supplement food is a food for the purpose of health supplementation. In some cases, the terms functional food, health food, and health supplement are used interchangeably with each other. To obtain a useful effect of the food on prevention or amelioration of obesity and diabetes, the food may be prepared in various forms such as tablets, capsules, powders, granules, liquids, pills, and the like.

Specific examples of these functional foods include processed foods that are modified to utilize the characteristics of agricultural products, livestock products, or fishery products using the zinc gluconate and cyclo-hispro or a sitologically acceptable salt thereof according to the present invention and that also have good storage properties.

The health functional food composition of the present invention may also be prepared in the form of nutritional supplements, food additives, feed, and the like, and fed to animals including humans or livestock.

The types of food compositions may be prepared in various forms according to a conventional method known in the art. General foods may be prepared by adding the zinc gluconate and cyclo-hispro or a sitologically acceptable salt thereof of the present invention to beverages (including alcoholic beverages), fruits and processed foods thereof (e.g., canned fruits, bottled foods, jam, marmalade, and the like), fish, meat and processed foods thereof (e.g., ham, sausage, corn, beef, and the like), bread and noodles (e.g., udon, buckwheat noodles, ramen, spaghetti, macaroni, and the like), fruit juices, various drinks, cookies, taffy, dairy products (e.g., butter, cheese, and the like), edible vegetable oils, margarine, vegetable proteins, retort foods, frozen foods, various seasonings (e.g., soybean paste, soy sauce, other sauces, and the like), and the like, but the present invention is not limited thereto.

Also, the nutritional supplements may be prepared by adding the zinc gluconate and cyclo-hispro or a sitologically acceptable salt thereof of the present invention to capsules, tablets, pills, or the like, but the present invention is not limited thereto.

In addition, as a health functional food, for example, the zinc gluconate and cyclo-hispro or a sitologically acceptable salt thereof of the present invention may be liquefied, granulated, encapsulated, and powdered, and then prepared into the form of tea, juice, or a drink so that the health functional food can be drunk as a health drink, but the present invention is not limited thereto. Also, the zinc gluconate and cyclo-hispro or a sitologically acceptable salt thereof of the present invention may be prepared into a powder or concentrate form for use in the form of food additives. In addition, the zinc gluconate and cyclo-hispro or a sitologically acceptable salt thereof of the present invention may be mixed with known active ingredients known to be effective in preventing or alleviating obesity and diabetes, and then prepared into the form of a composition.

When the food composition of the present invention is used as a health beverage composition, the health beverage composition may include various flavoring agents, natural carbohydrates, or the like as additional ingredients like conventional beverages. The above-described natural carbohydrates include monosaccharides such as glucose and fructose; disaccharides such as maltose and sucrose; polysaccharides such as dextrin and cyclodextrin; and sugar alcohols such as xylitol, sorbitol, erythritol, and the like. As sweeteners, natural sweeteners such as thaumatin and stevia extracts, and synthetic sweeteners such as saccharin and aspartame may be used. The proportion of the natural carbohydrates generally ranges from approximately 0.01 g to approximately 0.04 g, preferably approximately 0.02 g to 0.03 g, per 100 mL of the composition of the present invention.

The zinc gluconate and cyclo-hispro or a sitologically acceptable salt thereof of the present invention may be included as an active ingredient of the health functional food composition for preventing or ameliorating obesity and diabetes, and the amount thereof is an effective amount sufficient to obtain the prevention or amelioration effect, and preferably ranges from, for example, 0.01 to 100% by weight based on the total weight of the composition, but the present invention is not particularly limited thereto. The health functional food composition of the present invention may be prepared into the form of a composition by mixing the zinc gluconate and cyclo-hispro or a sitologically acceptable salt thereof with the active ingredients known to be effective in preventing or alleviating obesity and diabetes.

In addition to the above-listed ingredients, the health functional food of the present invention may include various nutrients, vitamins, electrolytes, flavoring agents, colorants, pectic acid and salts thereof, alginic acid and salts thereof, organic acids, protective colloidal thickening agents, pH adjusting agents, stabilizers, preservatives, glycerin, alcohols, carbonating agents, or the like. In addition, the health food of the present invention may include pulp for preparing natural fruit juices, fruit beverages, or vegetable beverages. These ingredients may be used alone or as a mixture thereof. The proportion of these additives is not very important, but is generally selected in the range of 0.01 to 0.1 parts by weight based on 100 parts by weight of the composition of the present invention.

A third aspect of the present invention relates to a method of preventing, ameliorating or treating obesity and diabetes in a subject in need thereof, which includes: administering to the subject an effective amount of a composition comprising the zinc gluconate and cyclo-hispro or a pharmaceutically acceptable salt thereof.

In the method of the present invention, the term “subject” includes any animal (e.g., a human, a horse, a pig, a rabbit, a dog, a sheep, a goat, a non-human primate, a cow, a cat, a guinea pig, or a rodent), but the present invention is not limited thereto. This term do not specify a specific age or sex. Accordingly, it is intended to include adult and neonatal subjects, whether female or male, as well as fetuses. The term “patient” refers to a subject suffering from a disease or disorder. The term “patient” includes human and veterinary subjects.

In the method of the present invention, the description of configurations including the effect, administration route, number of administrations, dosage, and the like of the zinc gluconate and cyclo-hispro or a pharmaceutically acceptable salt thereof administered to the subject is the same as described above and is therefore omitted.

In the method of the present invention, the zinc gluconate and cyclo-hispro may provide the desirable effect of preventing, ameliorating or treating obesity and diabetes when administered in effective amounts. For the desirable effect, the combination of zinc gluconate and cyclo-hispro or a pharmaceutically acceptable salt thereof of the present invention may be administered once or repeatedly several times at regular time intervals. At this time, the zinc gluconate and cyclo-hispro may be administered simultaneously, separately, or sequentially. For example, the zinc gluconate and cyclo-hispro or a pharmaceutically acceptable salt thereof may be co-formulated and administered simultaneously as a combined unit dosage preparation, or may be administered simultaneously or sequentially as separate preparations.

When administered sequentially, each of the active ingredients may be administered at any time intervals as an individual preparation, and the order of administration may be determined by a doctor or a person having ordinary knowledge in the art.

Also, the method of the present invention may be used in combination with other methods to prevent, ameliorate, or treat obesity and diabetes.

A fourth aspect of the present invention relates to a use of the composition, which includes the zinc gluconate and cyclo-hispro or a pharmaceutically acceptable salt thereof, for the manufacture of a medicament for preventing, ameliorating or treating obesity and diabetes.

In the use of the present invention, the description of configurations including the effect of the composition including the zinc gluconate and cyclo-hispro or a pharmaceutically acceptable salt thereof and the administration route, number of administrations, dosage, and the like thereof is the same as described above and is therefore omitted.

A fifth aspect of the present invention relates to a method of increasing β-nicotinamide adenine dinucleotide (NAD⁺) in a subject in need thereof, which includes: administering to the subject an effective amount of a composition comprising the zinc gluconate and cyclo-hispro or a pharmaceutically acceptable salt thereof.

In relation to the fifth aspect, the present invention provides a use of the composition, which includes the zinc gluconate and cyclo-hispro or a pharmaceutically acceptable salt thereof, for the manufacture of a β-nicotinamide adenine dinucleotide (NAD⁺) booster.

In the present invention, the increase in NAD⁺ may be an increase in the liver and/or visceral adipose tissue.

Here, the description of configurations including the effect of the composition, which includes the zinc gluconate and cyclo-hispro or a pharmaceutically acceptable salt thereof, and the administration route, number of administrations, dosage, and the like thereof is the same as described above and is therefore omitted.

A sixth aspect of the present invention relates to a method of enhancing Sirt1 deacetylase activity in a subject in need thereof, which includes: administering to the subject an effective amount of a composition comprising the zinc gluconate and cyclo-hispro or a pharmaceutically acceptable salt thereof.

In relation to the sixth aspect, the present invention provides a use of the composition, which includes the zinc gluconate and cyclo-hispro or a pharmaceutically acceptable salt thereof, for the manufacture of an Sirt1 deacetylase activator.

Here, the description of configurations including the effect of the composition including the zinc gluconate and cyclo-hispro or a pharmaceutically acceptable salt thereof and the administration route, number of administrations, dosage, and the like thereof is the same as described above and is therefore omitted.

Hereinafter, the present invention will be described in more detail with reference to embodiments thereof. However, it will be obvious to those skilled in the art that the following examples are provided only for illustration of the present invention and should not be construed as limiting the scope of the present invention.

### Example 1

### Confirmation of Obesity Improvement Effect According to Type of Zinc Salt

### 1-1. Experimental Animals and Drugs for Administration

Five-week-old male KKay mice from CLEA Japan Inc. were purchased through Saeron Bio Co., Ltd. Rearing was conducted under constant conditions (temperature: 22±2° C., relative humidity: 55±10%, circadian period: 12 hours), Purina rat feed was used as feed, and distilled water was used as drinking water. The mice were used in experiments after an adaptation period of one week after purchase. CHP was purchased from Angene Chemical Private Ltd., zinc gluconate was purchased from Captek Softgel International Inc., and zinc acetate, zinc chloride, and zinc sulfate were purchased from Sigma-Aldrich.

The five-week-old KKay mice were preliminarily reared for one week and then randomly divided into groups so that their average body weights were the same. The administration conditions for each group are shown in Table 1. Distilled water was orally administered daily to the control. Gluconate-CycloZ in which zinc gluconate and CHP were mixed in a weight ratio of 2:1 (based on the zinc element) was orally administered once a day to experimental group 1, acetate-CycloZ in which zinc acetate and CHP were mixed in a weight ratio of 2:1 (based on the zinc element) was orally administered once a day to experimental group 2, chloride-CycloZ in which zinc chloride and CHP were mixed in a weight ratio of 2:1 (based on the zinc element) was orally administered once a day to experimental group 3, and sulfate-CycloZ in which zinc sulfate and CHP were mixed in a weight ratio of 2:1 (based on the zinc element) was orally administered once a day to experimental group 4. Thereafter, the body weights were measured weekly.

TABLE 1
Group setting and drug administration

| Group | Administered drug |
|---|---|
| Control (CTRL) | Distilled water |
| Experimental group 1 | Zinc gluconate (70 mg/kg) + CHP |
| (Gluconate-CycloZ, 75 mg/kg) | (5 mg/kg) |
| Experimental group 2 | Zinc acetate (33 mg/kg) + CHP |
| (Acetate-CycloZ, 38 mg/kg) | (5 mg/kg) |
| Experimental group 3 | Zinc chloride (21 mg/kg) + CHP |
| (Chloride-CycloZ, 26 mg/kg) | (5 mg/kg) |
| Experimental group 4 | Zinc sulfate (44 mg/kg) + CHP |
| (Sulfate-CycloZ, 49 mg/kg) | (5 mg/kg) |

### 1-2. Confirmation of Weight Loss Effect

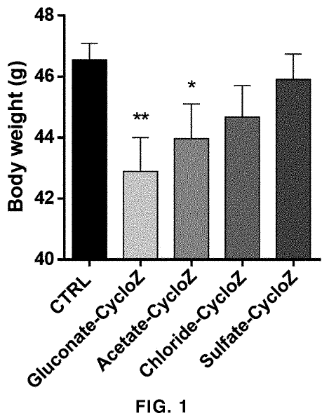

Body weights were measured every week for 15 weeks of drug administration, and the body weights were compared at week 15. As a result, as can be seen in FIG. 1, it was confirmed that the average body weight, which was approximately 26.6 g at the start of this experiment, increased to 46.6 g in the case of the control group, whereas the average body weight was decreased at 42.9 g, 44 g, 44.7 g, and 45.9 g in the cases of experimental group 1, experimental group 2, experimental group 3, and experimental group 4, respectively. It was confirmed that the weight loss effects compared to the control were 7.9%, 5.6%, 4.1%, and 1.5% in that order in the experimental groups, depending on the type of zinc salt, and the reduction effect and significance were greatest in experimental group 1. Accordingly, it was confirmed that gluconate-CycloZ containing zinc gluconate was most effective in ameliorating obesity compared to CycloZ containing other zinc salts.

### Example 2

### Confirmation of Diabetes Improvement Effect According to Type of Zinc Salt

### 2-1. Oral Glucose Tolerance Test and Glycated Hemoglobin Measurement

To confirm the diabetes improvement effect according to the type of zinc salt, an oral glucose tolerance test and glycated hemoglobin measurement were performed. First, for an oral glucose tolerance test (OGTT), mice were fasted for 16 hours and 2 g/kg of glucose was administered by oral gavage. Blood was collected from the tail vein at 15, 30, 60, 90, and 120 minutes after glucose administration. Blood glucose levels were immediately measured using a blood glucose meter (AGM-4000, Allmedicus, Anyang, Korea). Blood was collected from the tail vein to measure glycated hemoglobin (HbA1c). HbA1c was measured using a DCA Vantage® analyzer (Siemens, Munich, Germany).

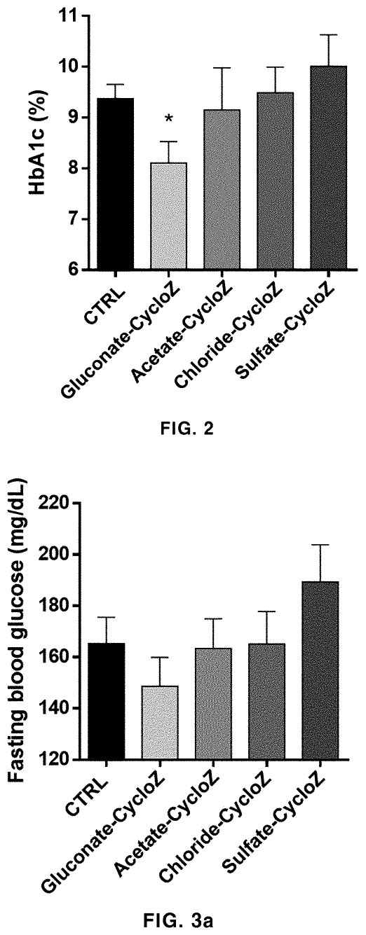

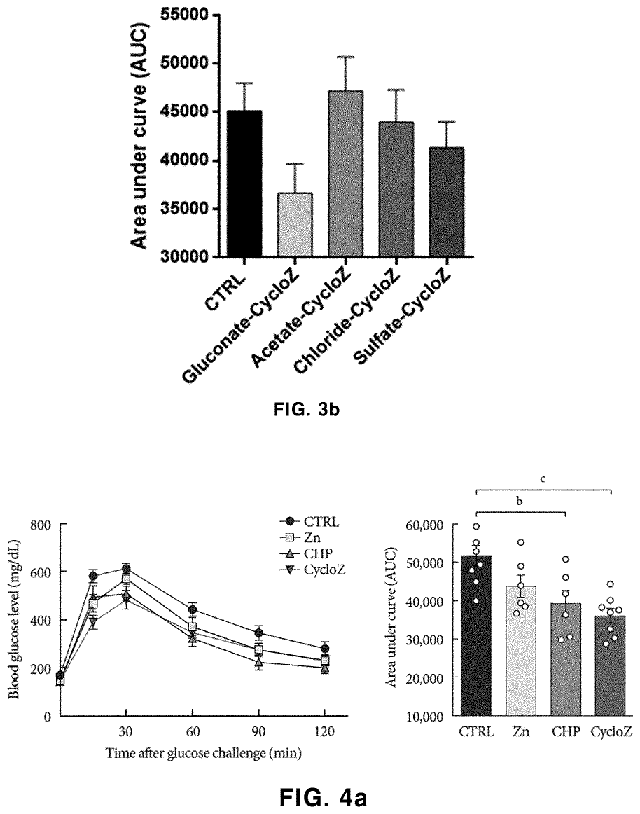

As shown in FIG. 2, the level of glycated hemoglobin in the blood, which is a standard indicator of diabetes, showed a decrease of approximately 13.5% in glycated hemoglobin compared to the control, and significance was confirmed only in experimental group 1. Also, as shown in FIGS. 3A and 3B, it was confirmed that a decrease in fasting blood glucose was observed in experimental group 1, and blood glucose decreased most rapidly for 2 hours after glucose administration. The glucose tolerance test values decreased by 18.8% (experimental group 1), 2.5% (experimental group 2), and 8.4% (experimental group 3) depending on the type of zinc salt compared to the control group. In experimental group 4, there was no reduction effect compared to the control.

Based on these results, it was confirmed that gluconate-CycloZ containing zinc gluconate was most effective compared to CycloZ containing other zinc salts in controlling blood glucose and ameliorating diabetes.

### Statistical Analysis

The statistical significance of the experimental results of Examples 1 and 2 was analyzed for each experimental group using the t-test statistical method against the control. *p<0.05, **p<0.01.

### Example 3

Confirmation of T2DM and Obesity Improvement Effects with Gluconate-CycloZ Administration in Animal Model of Diabetes and Obesity

### 3-1. Experimental Animals and Drugs for Administration

Five-week-old male KK-Ay mice purchased from CLEA Japan Inc. (Nishi-Shimbashi, Japan) were reared in individual cages in an air-conditioned room at a temperature of 23±3° C. with a 12-hour light/dark cycle. Distilled water and laboratory diet were freely available. All animal experiments were approved in accordance with the Ethical Review Committee of the Pohang Advanced Bio Convergence Center, Korea (ABCC201712). All animals were used in the experiments after 1 week of adaptation.

For the prevention studies, the animals were divided into two groups. As an excipient, water was administered to the control. Gluconate-CycloZ (CHP: 5 mg/kg and zinc gluconate: 70 mg/kg) was supplied to the KK-Ay mice by oral gavage daily for 20 weeks. For treatment studies, the animals were divided into the control and experimental groups at 12 weeks of age. Water or gluconate-CycloZ was supplied to the KK-Ay mice by oral gavage daily for 8 weeks. At the end of each experiment, all the mice were anesthetized with isoflurane using the RC2 Rodent Circuit Controller Anesthesia system (Vetequip, Pleasanton, CA, USA). Blood was collected through cardiac puncture, and plasma was separated. Separated adipose tissue, liver, and plasma were stored at −80° C. until analysis.

### 3-2. Oral Glucose Tolerance Test and Glycated Hemoglobin Measurement

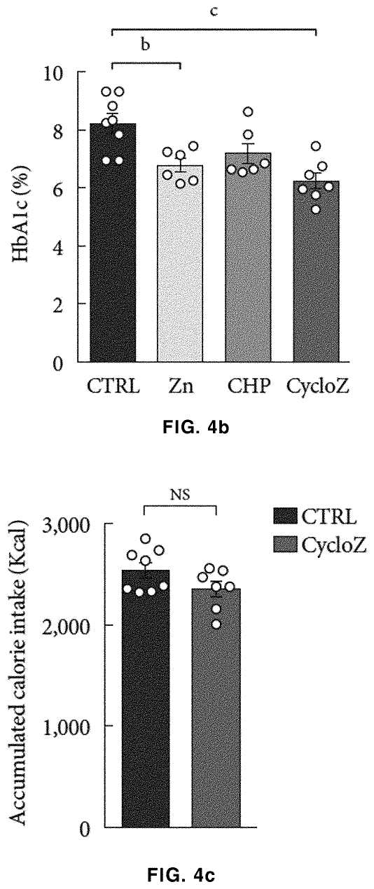

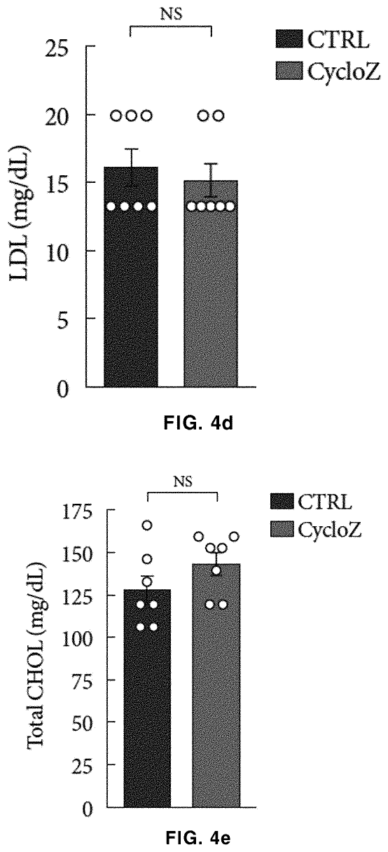

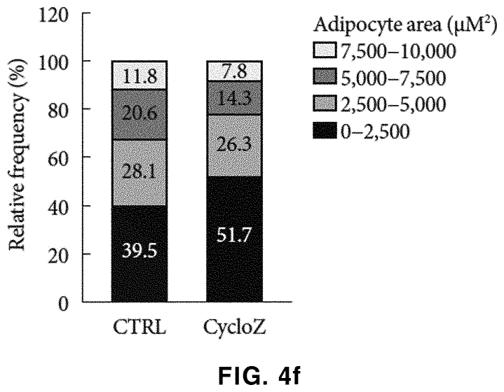

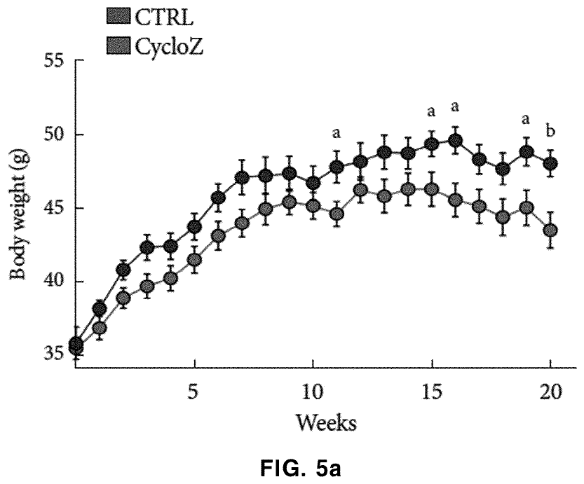

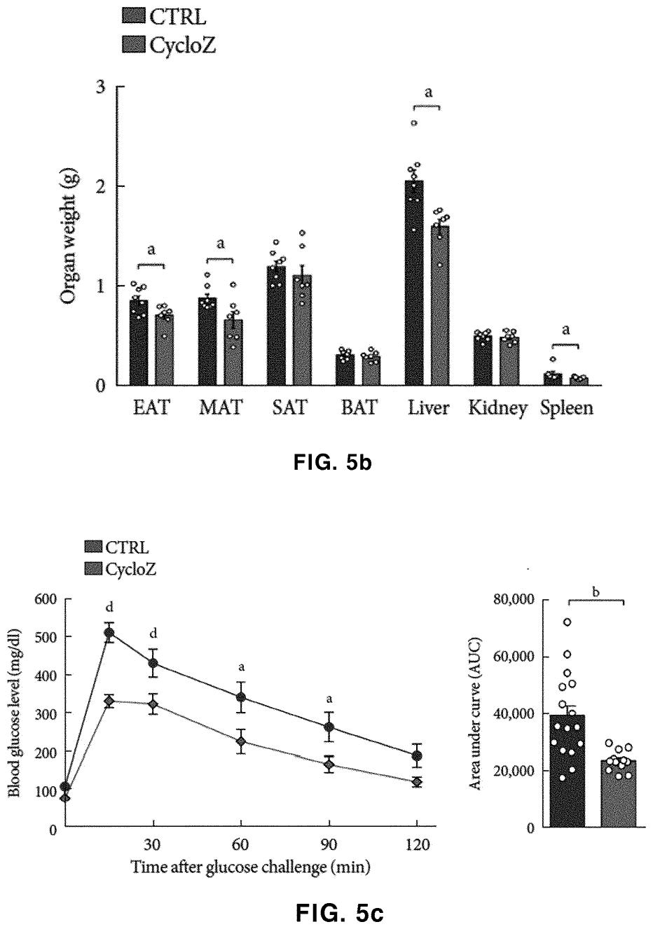

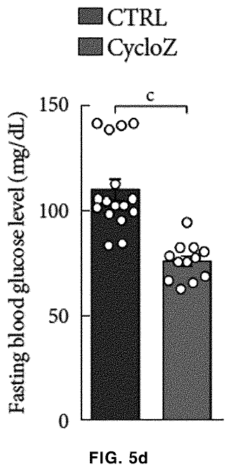

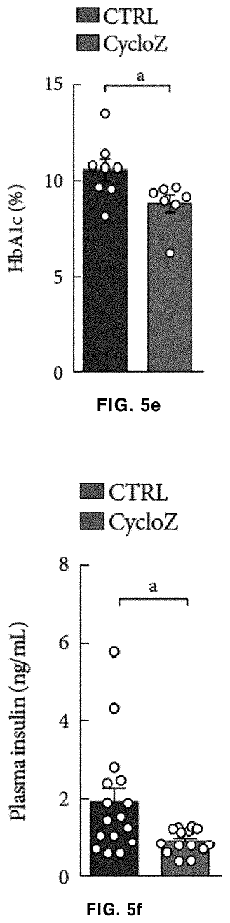

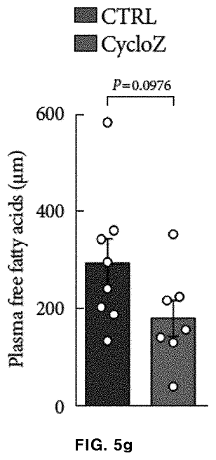

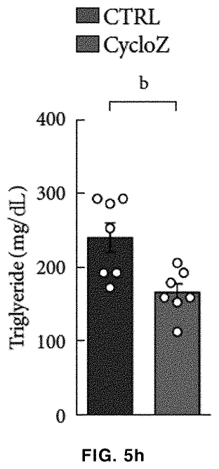

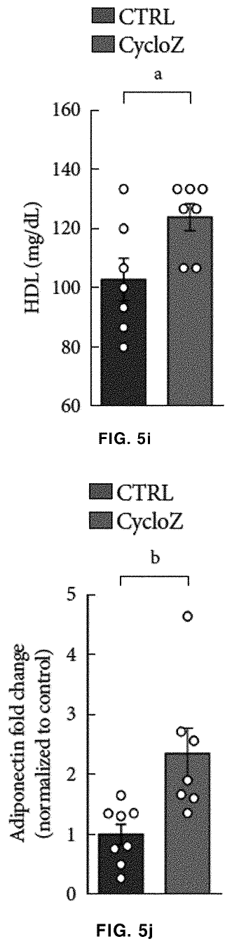

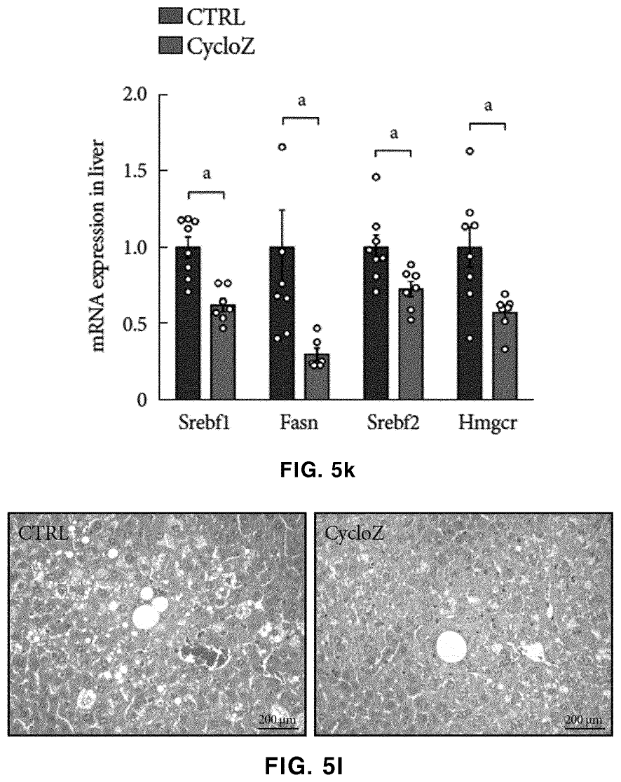

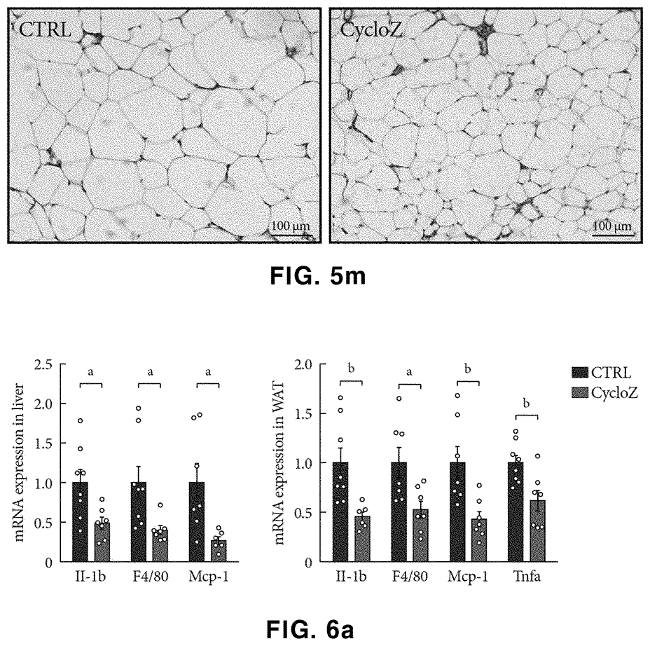

An oral glucose tolerance test and glycated hemoglobin measurement were performed in the same manner as in Example 2-1. As shown in FIGS. 4A and 4B, the results showed that the gluconate-CycloZ administration was more effective in improving glucose tolerance compared to the treatment with individual compounds. Also, it was confirmed through FIG. 5C that gluconate-CycloZ improved the glucose tolerance of KK-Ay mice as measured according to the results of OGTT. As shown in FIGS. 5D, 5E, and 5F, it was confirmed that fasting blood glucose, HbA1c levels, and plasma insulin concentrations were significantly reduced by gluconate-CycloZ administration. These results demonstrate that gluconate-CycloZ improves glucose metabolism and insulin sensitivity.

### 3-3. Body Weight, Food Intake, and Organ Weight Measurements

As shown in FIGS. 5A and 4C, the weight gain of mice in the gluconate-CycloZ-treated group gradually decreased compared to the control at the end of the experiment, and there was no noticeable difference in food intake. Also, as shown in FIG. 5B, the gluconate-CycloZ administration significantly suppressed an increase in mass of liver and visceral adipose tissues (VAT) such as epididymal adipose tissue (EAT), mesenteric adipose tissue (MAT), and the like, but did not suppress an increase in mass of subcutaneous adipose tissue. The treatment with gluconate-CycloZ also reduced liver and VAT weights in the mice.

### 3-4. Analysis of Blood Biochemical Parameters

Whole blood was collected through cardiac puncture, and centrifuged at 2,000xg for 10 minutes to separate plasma. Thereafter, the plasma was stored at −80° C. until analysis. Aspartate aminotransferase (AST), alanine aminotransferase, alkaline phosphatase, total cholesterol, high-density lipoprotein cholesterol (HDL-C), low-density lipoprotein cholesterol, creatinine, and blood urea nitrogen (BUN) were measured using a biochemical analyzer (BS-390, Mindray Bio-medical Electronics Co. Ltd., Shenzhen, China). Free fatty acids were quantified using an Enzy-Chrom Free Fatty Acid Assay kit (EFFA-100, BioAssay System, Hayward, CA, USA).

As shown in Table 2, it can be seen that the gluconate-CycloZ administration did not cause changes in AST, BUN, and creatinine, indicating that gluconate-CycloZ possessed superior safety and tolerance.

TABLE 2

| | Control | Gluconate-CycloZ |
|---|---|---|
| AST (U/L) | 121.97 ± 30.19 | 111.48 ± 26.37 (P = 0.5339) |
| ALP (U/L) | 32.40 ± 8.31 | 22.87 ± 7.00 (P = 0.0528) |
| BUN (mg/dL) | 26.20 ± 3.4 | 26.11 ± 2.93 (P = 0.9632) |
| Creatinine (mg/dL) | 2.15 ± 0.17 | 2.08 ± 0.17 (P = 0.4508) |

The blood lipid profile of the mice was also examined using a biochemical analyzer. As shown in FIGS. 5G, 5H, and 5I, in the mice treated with gluconate-CycloZ, the free fatty acid and triglyceride levels decreased and the HDL-C level increased. However, as shown in FIGS. 4D and 4E, it was observed that the total cholesterol levels remained unchanged.

3-5. RNA Extraction, cDNA Synthesis, and mRNA Expression Analysis

Total RNA was extracted from tissues and cells using a NucleoZOL reagent (740404.200, Macherey-Nagel, Allentown, PA, USA). 1 μg of total RNA was used for cDNA synthesis using an iScript cDNA synthesis kit (1708891, Bio-Rad, Hercules, CA, USA). Real-time quantitative polymerase chain reaction (RT-qPCR) was performed using the gene-specific primers in Table 3 and IQ SYBR Green Supermix (BR1708882, Bio-Rad). RT-qPCR was performed with the following amplification reaction cycles: 95° C. for 10 seconds, 60° C. for 10 seconds, and 72° C. for 30 seconds. Expression levels were normalized to that of β-actin or glyceraldehyde-3-phosphate dehydrogenase (GAPDH).

TABLE 3

| | | SEQ | | SEQ |
|---|---|---|---|---|
| | Forward | ID | Reverse | ID |
| | (5′ → 3′) | NO: | (5′ → 3′) | NO: |
| Srebf1 | CGACTACATCCGCT | 1 | GGTCCTTCAGTGAT | 2 |
| | TCTTG | | TTGCTT | |
| Fasn | AATGCTGCTCTTGA | 3 | AAAGAGACTGAACC | 4 |
| | TGCTCTC | | GAAGGCA | |
| Srebf2 | GCGACCAGGAAGAA | 5 | ACAAATCCCACAGA | 6 |
| | GAGA | | GTCCA | |
| Hmgcr | GTAAGCGCAGTTCC | 7 | CGGATCTCAATGGA | 8 |
| | TTCCGC | | GGCCAAG | |

The reduction of lipid levels in Example 3-4 was explained by decreased mRNA expression of genes involved in the fatty acid and cholesterol synthesis, including sterol regulatory-element binding transcription factor 1 (Srebf1), fatty acid synthase (Fasn), Srebf2, and 3-hydroxy-3-methylglutaryl-CoA reductase (Hmgcr), as shown in FIG. 5K.

### 3-6. Histochemistry

Liver and adipose tissues were fixed in a neutral buffered 10% formalin solution (HT-501128, Sigma, St. Louis, MO, USA) and embedded into paraffin blocks. A series of (4 μm thick) sections were deparaffinized, dehydrated, and then stained with hematoxylin and eosin (H&E). Immunohistochemical analysis was performed using antibodies against tumor necrosis factor a (TNFα, Abcam, Cambridge, UK; ab1793), macrophage chemoattractant protein 1 (MCP-1, Abcam, ab25124), F4/80 (Abcam, ab111101), and CD11b (Abcam; ab133357). Stained areas were observed under a light microscope (Olympus BX53 upright microscope, Olympus, Tokyo, Japan).

As shown in FIG. 5L, it was confirmed that the gluconate-CycloZ administration also improved hepatic lipid deposition, whereas the control showed fatty liver pathology such as steatosis. Also, as shown in FIGS. 5M and 4F, it was confirmed that the area of adipocytes in EAT was significantly reduced in the gluconate-CycloZ-treated group compared to the control.

### 3-7. Western Blot

For protein experiments, tissues and cells were lysed with a radioimmunoprecipitation assay (RIPA) buffer (89901, Thermo Scientific, Waltham, MA, USA) including a cocktail of Halt protease and phosphatase inhibitors (78440, Thermo Scientific). Bolt 4%-12% Bis-Tris Plus Gel (Thermo Scientific) was used for SDS-PAGE, and transfer was performed using a Trans-Blot Turbo system (Bio-Rad). The following antibodies were used to detect target proteins: PGC-1α (NBP1-04676, Novusbio, Centennial, CO, USA), Ac-Lysine (9814S, Cell Signaling Technology [CST], Danvers, MA, USA), phospho-AMPK (5831S, CST), AMPK (2535S, CST), phospho-Akt (9271S, CST), Akt (9272S, CST), LC3 I/II (4108S, CST), GAPDH (2118S, CST), adiponectin (2789S, CST), and Sirt1 (07-131, EMD Millipore, Burlington, MA, USA).

As shown in FIG. 5J, it was confirmed that the adiponectin levels significantly increased in the blood from the gluconate-CycloZ-treated mice.

Based on the above results, it was confirmed that the weight loss caused by gluconate-CycloZ administration was due to fat reduction through regulation of the lipid and cholesterol metabolism in the liver and VAT.

### Example 4

Confirmation of Inflammation Improvement and Immune Cell Infiltration Reduction Effects with Gluconate-CycloZ Administration in Animal Model of Diabetes and Obesity
4-1. mRNA Expression Analysis

Because obesity-induced insulin resistance in the liver and VAT is closely associated with chronic inflammation in many tissues, the present inventors examined through mRNA expression analysis that gluconate-CycloZ reduced the production of proinflammatory cytokines (TNFα and MCP-1) and monocyte infiltration (F4/80 and CD11b). RT-qPCR was performed in the same manner as in Example 3-5, and information on the gene-specific primers used is shown in Table 4.

TABLE 4

| | | SEQ | | SEQ |
|---|---|---|---|---|
| | Forward | ID | Reverse | ID |
| | (5′ → 3′) | NO: | (5′ → 3′) | NO: |
| Il1B | CTCAACTGTGAAATG | 9 | CTTCATCTTTTGGG | 10 |
| | CCACC | | GTCCGT | |
| F4/80 | AGACGGCTTGTGCCA | 11 | CTGCCTCCACTAGC | 12 |
| | TCATT | | ATCCAG | |
| Mcp1 | GCTACAAGAGGATCA | 13 | GCACAGACCTCTCT | 14 |
| | CCAGCA | | CTTGAGC | |
| Tnfa | TGTCTACTCCCAGGT | 15 | ATAGCAAATCGGCT | 16 |
| | TCTCTT | | GACGG | |

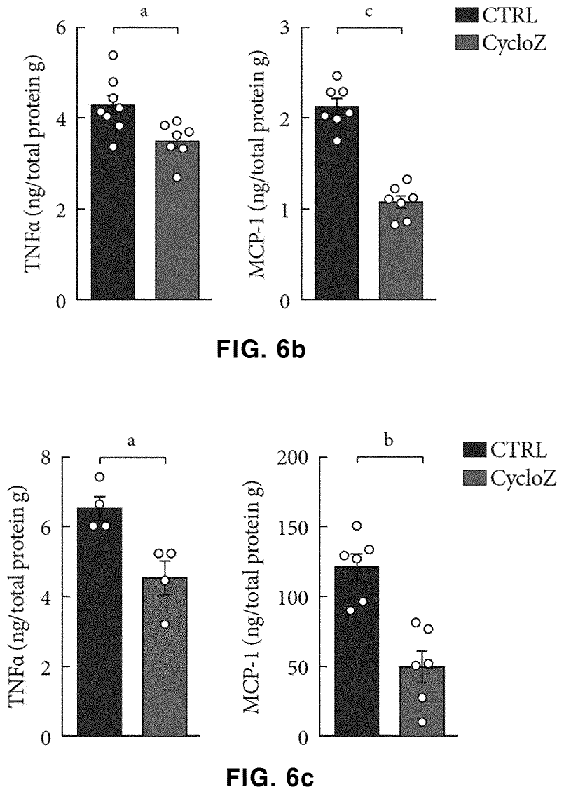

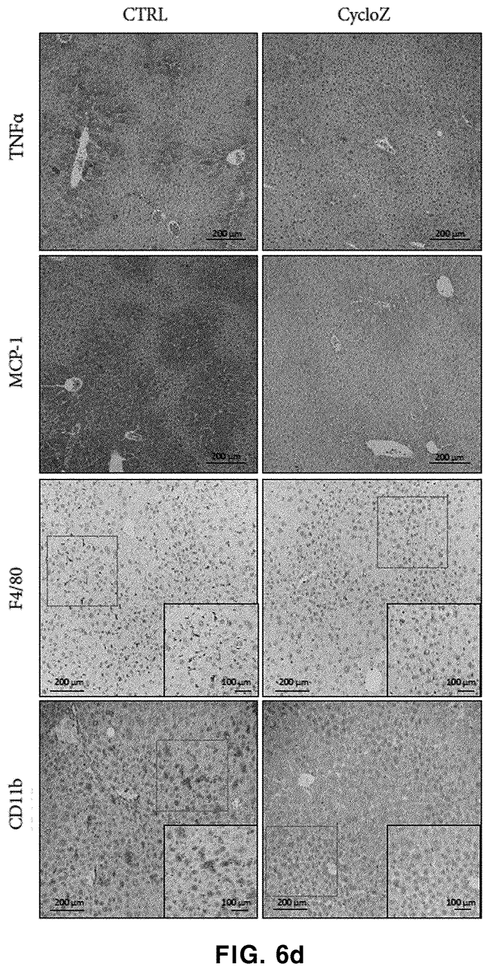

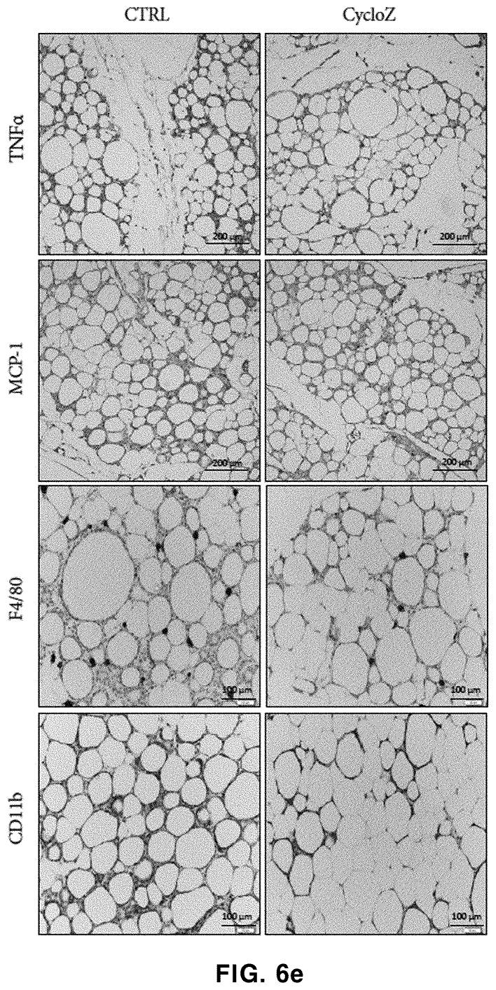

The expression of inflammatory cytokine genes in the liver and MAT and the expression levels of F4/80 and MCP-1 were significantly reduced by gluconate-CycloZ administration, as shown in FIG. 6A. Consistently, it can be seen through FIGS. 6B and 6C that the treatment with gluconate-CycloZ also caused a significant decrease in TNFα and MCP-1 protein levels in the liver and EAT.

### 4-2. Histochemistry

The expression of TNFα, MCP-1, F4/80, and CD11b in the liver and EAT was examined by immunohistochemistry. Histochemistry was performed in the same manner as Example 3-6. As shown in FIGS. 6D and 6E, it was confirmed that the production of inflammatory cytokines and the monocyte infiltration both disappeared with gluconate-CycloZ administration. In summary, these results suggest that the improvement in tissue insulin resistance after gluconate-CycloZ administration was accompanied by a decrease in inflammation.

### Example 5

Confirmation of Mitochondrial Biogenesis and Inflammation Improvement Effects with Gluconate-CycloZ Administration in Animal Model of Diabetes and Obesity

### 5-1. Western Blot

Infiltrating macrophages play an important role in the development of insulin resistance in metabolic organs. Their activity is partially regulated by deacetylation of transcription factors such as a p65 subunit of NF-κB. Also, lysine acetylation regulates the activities of many metabolic enzymes and transcription factors. Previous reports revealed that the overall lysine acetylation profile increases in the kidney and heart of diabetic patients (Berthiaume, Jessica M., et al. “Methylene blue decreases mitochondrial lysine acetylation in the diabetic heart,” Molecular and Cellular Biochemistry 432 (2017): 7-24; and Kosanam, Hari, et al. “Diabetes induces lysine acetylation of intermediary metabolism enzymes in the kidney,” Diabetes 63.7 (2014): 2432-2439). Therefore, in this example, Western blot was performed in the same manner as in Example 3-7 to investigate the total acetyl lysine level in the liver of mice in the gluconate-CycloZ-treated group.

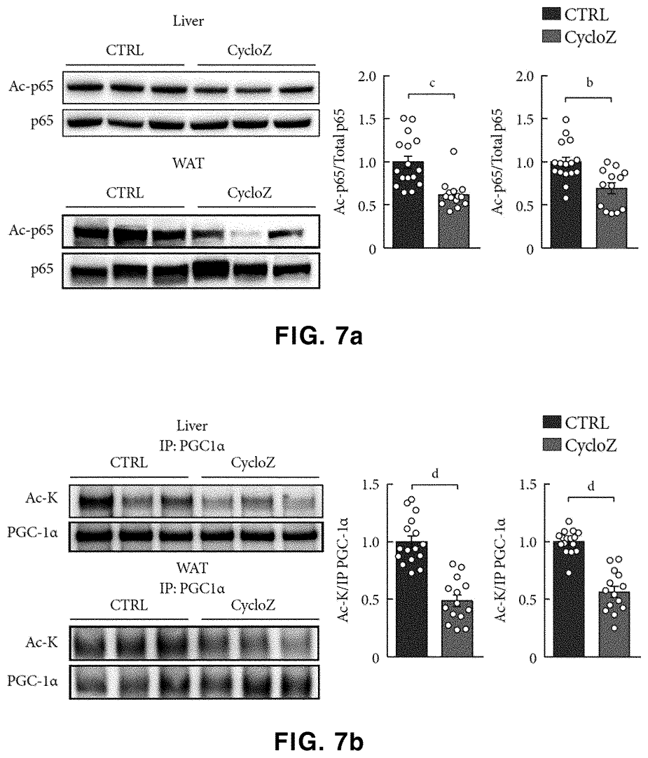

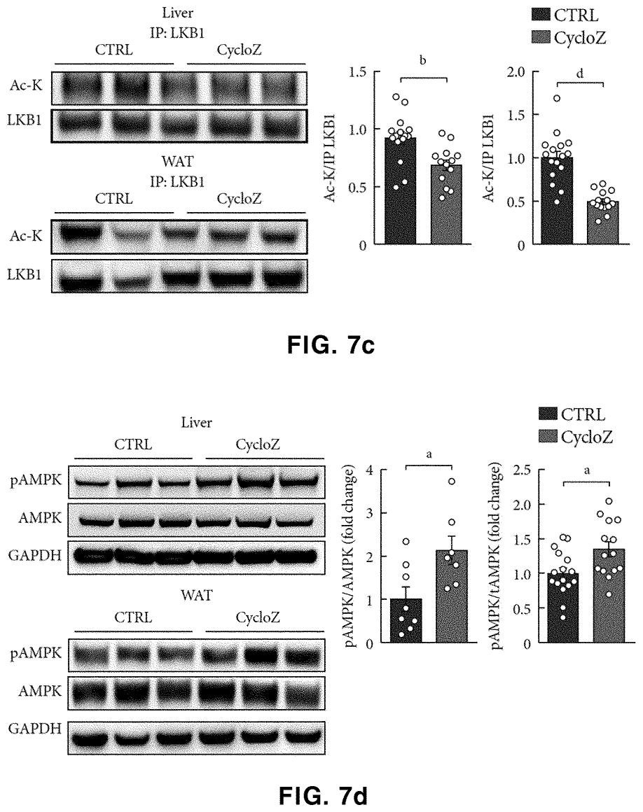

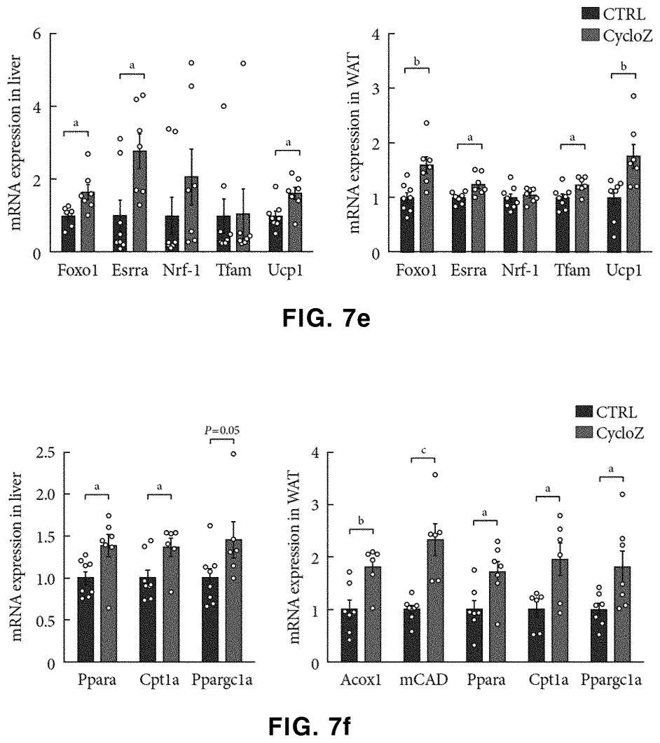

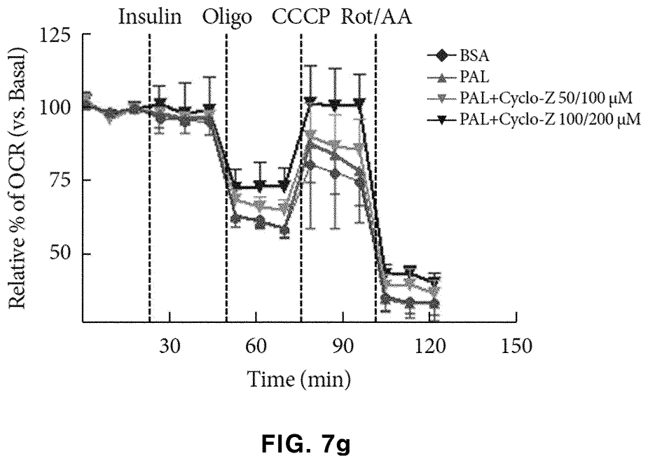

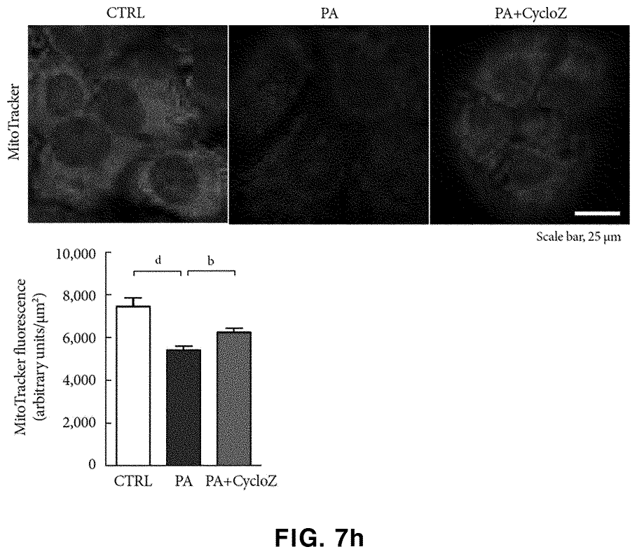

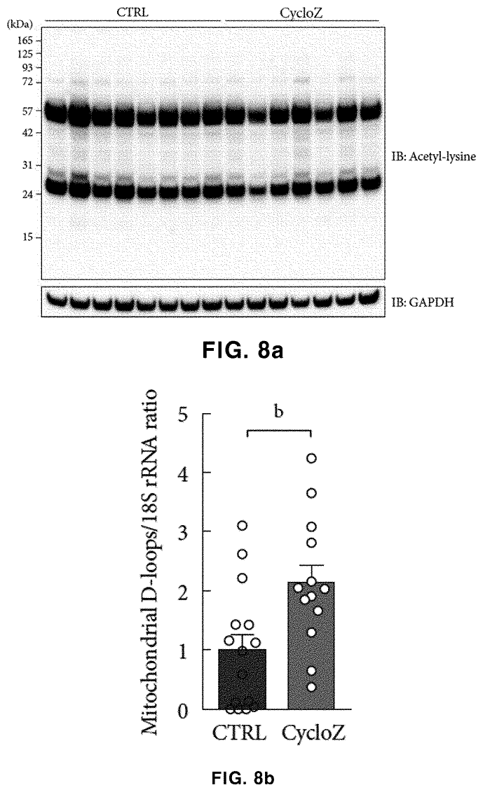

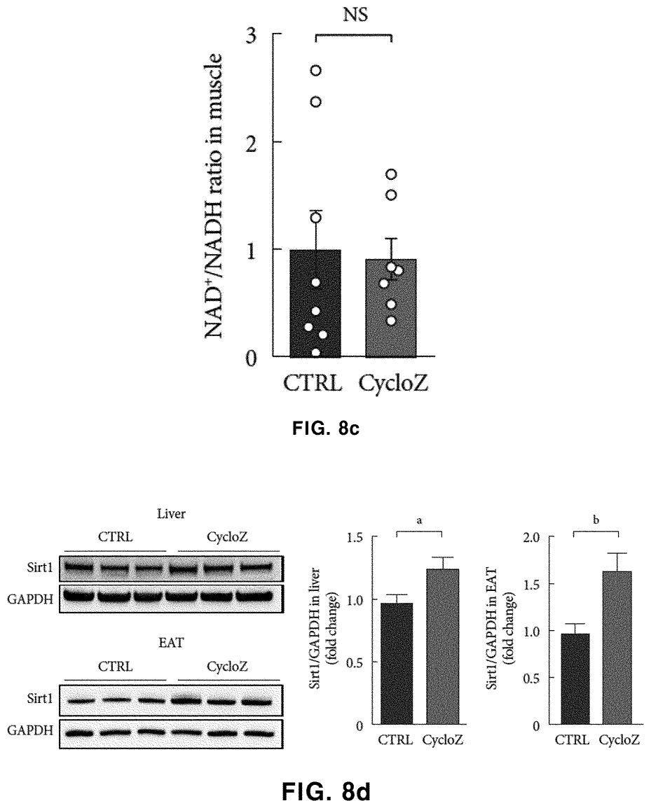

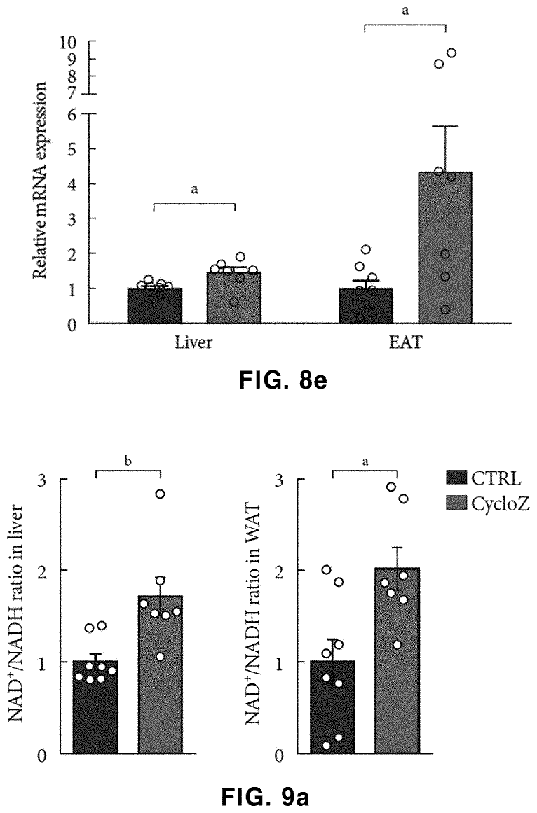

As shown in FIG. 7A, it can be seen that the acetylation of p65 was strongly reduced in the liver and EAT of the gluconate-CycloZ-treated mice. Also, as shown in FIG. 8A, it was confirmed that the total acetyl lysine level in the liver of mice in the gluconate-CycloZ-treated group was significantly reduced compared to the level in the control. Moreover, as shown in FIGS. 7B and 7C, it was confirmed that the acetylation of PGC-1α and LKB1 was significantly reduced in the liver and EAT of the gluconate-CycloZ-treated mice.

In addition to the deacetylation, PGC-1α requires AMPK-mediated phosphorylation for activation. Therefore, to investigate whether CycloZ affects the AMPK-PGC-1α pathway, Western blot was also performed in the same manner as in Example 3-7. As a result, as shown in FIG. 7D, it was confirmed that the AMPK phosphorylation increased in the liver and EAT of gluconate-CycloZ-treated mice.

5-2. mRNA Expression Analysis Next, to investigate the expression of PGC-1α-related genes, RT-qPCR was performed in the same manner as in Example 3-5, and information on the gene-specific primers used is shown in Table 5.

TABLE 5

| | | SEQ | | SEQ |
|---|---|---|---|---|
| | Forward | ID | Reverse | ID |
| | (5′ → 3′) | NO: | (5′ → 3′) | NO: |
| Foxo1 | CCTTTCCTCCTCCC | 17 | TGCCTCTACTGAAT | 18 |
| | TCTG | | GATTACAC | |
| Esrra | CAGGAGGCAGACAC | 19 | CGGATTAAGCAGCA | 20 |
| | TGAT | | GCAA | |
| Nrf1 | CCTCAGCCTCCATC | 21 | GACCTTACAACCAA | 22 |
| | TTCT | | GCAACT | |
| Tfam | CGGACCTCTAAGAT | 23 | CTACCTTTCCCATT | 24 |
| | CTAACTAC | | CCCTTC | |
| Ucp1 | | 25 | | 26 |
| Ppara | ACTTGCCTCACTAC | 27 | TGCTGGTATCGGCT | 28 |
| | TGTCCTT | | CAATA | |
| Cpt1a | CTGGATGTTTGCAG | 29 | TCGACCCGAGAAGA | 30 |
| | AGCACG | | CCTTGA | |
| Ppargc1a | TGGAGTGACATAGA | 31 | TCAGAAAGGTCAAG | 32 |
| | GTGTGCTG | | TTCAGGAAGA | |
| Acox1 | ACACTAACATATCA | 33 | CATTGCCAGGAAGA | 34 |
| | ACAAGAGGAG | | CCAG | |
| Mcad | TAGACGAAGCCACG | 35 | GAGCCTAGCGAGTT | 36 |
| | AAGTA | | CAAC | |

As shown in FIG. 7E, it can be seen that the expression of forkhead box O1 (Foxo1), estrogen-related receptor alpha (Esrra), transcription factor A, mitochondrial (Tfam), nuclear respiratory factor 1 (Nrf1), and uncoupling protein 1 (Ucp1), which are required for mitochondrial biogenesis, increased in the liver and MAT of the mice in the gluconate-CycloZ-treated group compared to the control. Similarly, as shown in FIG. 7F, the expression of peroxisome proliferator-activated receptor alpha (Ppara), carnitine palmitoyltransferase 1A (Cpt1a), and carnitine palmitoyltransferase 1A (Ppargc1a), which are related to lipid oxidation in the liver, also increased in the gluconate-CycloZ-treated group, and the acyl-CoA oxidase 1 (Acox1), medium-chain acyl-CoA dehydrogenase (Mcad), Ppara, Cpt1a, and Ppargc1a levels increased in MAT of the gluconate-CycloZ-treated mice.

Increased mitochondrial biogenesis upon gluconate-CycloZ administration was evidenced by increased mitochondrial DNA (mtDNA) content in the livers of the gluconate-CycloZ-treated mice, as shown in FIG. 8B. This means that gluconate-CycloZ improves mitochondrial biogenesis and lipid oxidation activation, which increases the mitochondrial function.

### 5-3. Measurement of Oxygen Consumption Rate

Mitochondrial respiration and OCR in AML12 mouse hepatocytes were confirmed using the following methods. An oxygen consumption rate (OCR) was measured using an XF96 extracellular flux analyzer (Seahorse Bioscience, Billerica, MA, USA) according to the manufacturer's protocol. AML12 cells were seeded in an XF-96 tissue culture plate at a density of 1×10⁴ cells/well. The next day, the medium was replaced with an XF basic medium (pH 7.4, Seahorse Biosciences, North Billerica, MA, USA) supplemented with 25 mM D-glucose (G7528, Sigma-Aldrich), 1 mM sodium pyruvate (S8636, Sigma-Aldrich), and 1 X GlutaMAX™ (35050, Gibco, Waltham, MA, USA), and then treated with an appropriate drug. To evaluate OCR, the compounds and metabolites used in this example were as follows: insulin (100 nM, I5556, Sigma-Aldrich), oligomycin A (1 μM, 75351, Sigma-Aldrich), carbonyl cyanide 4-(trifluoromethoxy) phenylhydrazone (CCCP, 2 μM, C2920, Sigma-Aldrich), and rotenone (1 μM, R8875, Sigma-Aldrich). To normalize to the cell number, 4′,6-diamino-2-phenylindole (DAPI)-stained cells were automatically counted using ImageXpress Micro Confocus Microscopy (Molecular Devices, San Jose, CA, USA).

As shown in FIG. 7G, it was confirmed that the OCR, ATP-linked respiration, and maximal respiratory capacity were significantly improved in the gluconate-CycloZ-treated group compared to the palmitate-treated group.

### 5-4. MitoTracker Staining

0.5×10⁵ cells were plated on a glass coverslip in a 12-well plate and cultured for 24 hours. 200 μM palmitate was treated in serum-free medium in the presence or absence of gluconate-CycloZ for 24 hours. The cells were treated with 500 nM MitoTracker Deep Red FM (M22426, Invitrogen, Carlsbad, CA, USA) diluted in a serum-free medium, and incubated for 30 minutes. The cells were washed with PBS and fixed in 4% paraformaldehyde at room temperature for 40 minutes. After Hoechst staining, the coverslip was mounted on a glass slide. Images (7 to 10) of each group were taken using a Leica confocal laser scanning microscope. The fluorescence of five randomly selected regions in each image was measured using a Leica LAS AF program (Leica, Wetzlar, Germany).

MitoTracker Deep Red FM staining results in FIG. 7H showed that the mitochondrial mass increased in gluconate-CycloZ-treated AML12 cells. These results show that gluconate-CycloZ improves the mitochondrial biogenesis and function by regulating the acetylation status.

### Example 6

Confirmation of NAD⁺ Synthesis Increase Effect with Gluconate-CycloZ Administration in Animal Model of Diabetes and Obesity

Deacetylation of non-histone transcription factors PGC-1α, LKB1, and p65 was observed in the liver and VAT. The sirtuin family of deacetylases is known to regulate acetylation of the proteins. Sirt1 has the broadest range of substrates and affects a variety of physiological pathways, including energy metabolism. Because Sirt1 uses NAD⁺ as a co-substrate to remove acetyl groups, the cellular NAD⁺/NADH ratio reflects the Sirt1 enzymatic activity. Therefore, the present inventors hypothesized that gluconate-CycloZ increases the deacetylation activity of Sirt1 by increasing the NAD⁺ levels or NAD⁺/NADH ratio.

6-1. NAD⁺/β-nicotinamide adenine dinucleotide Quantification

NAD⁺ and β-nicotinamide adenine dinucleotide (NADH) ratios were measured in a tissue lysate using an NAD⁺/NADH quantitative colorimetric kit (K-337-100, Biovision, Milpitas, CA, USA) according to the manufacturer's protocol. In summary, 10 mg of tissue was homogenized with the provided extraction buffer. To measure the total NAD⁺ concentration, 50 μL of the extracted sample was transferred to a 96-well microplate. To decompose NAD, the remaining extracted sample was heated at 60° C. for 30 minutes. As a result, 50 μL of the decomposed sample was transferred to a 96-well microplate. After development, the plate was measured at 450 nm.

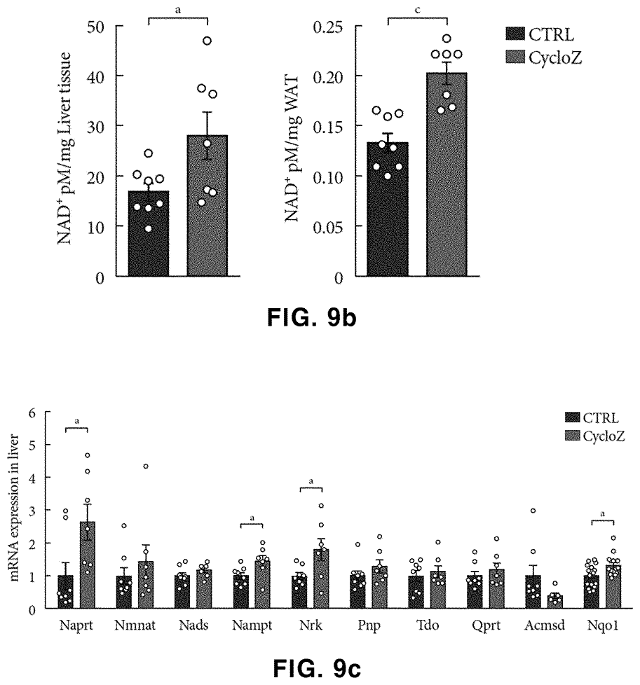

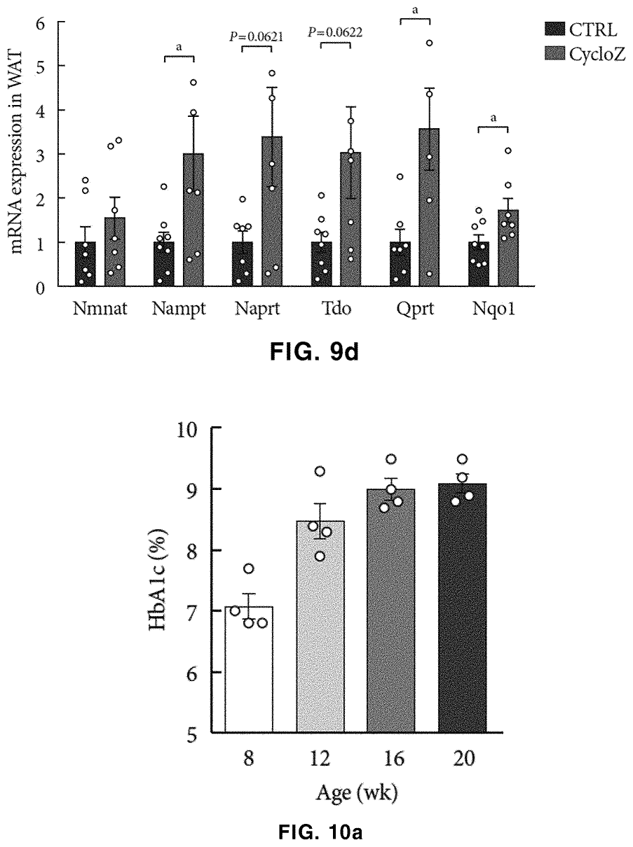

As shown in FIGS. 9A and 8C, it was found that the NAD⁺/NADH ratio increased in the liver and EAT with gluconate-CycloZ administration, but did not increase in the muscle. It can be seen through FIG. 9B that the increase in NAD⁺/NADH ratio in the liver and EAT is due to the increase in the total amount of NAD⁺.

6-2. mRNA Expression Analysis

To investigate why the NAD⁺ level increased, the expression of genes involved in the NAD⁺ synthesis was confirmed by RT-qPCR. The experimental method was the same as Example 3-5, and information on the gene-specific primers used is shown in Table 5.

TABLE 6

| | | SEQ | | SEQ |
|---|---|---|---|---|
| | Forward | ID | Reverse | ID |
| | (5′ → 3′) | NO: | (5′ → 3′) | NO: |
| Naprt | GCCCTCCTTTCGTG | 37 | CACAGGCTCCCGAC | 38 |
| | TGAGTT | | TAATGG | |
| Nmnat | ATTGCTGTGTGGGG | 39 | CCACGATTTGCGTG | 40 |
| | CAGATT | | ATGTCC | |
| Nads | CCCTGGACTTTGAG | 41 | CCAAGCCTGTATCT | 42 |
| | GGCAAT | | TGCACC | |
| Nampt | AAGAGACTGCTGGC | 43 | TTAGAGCAATTCCC | 44 |
| | ATAGGG | | GCCACA | |
| Nrk | GGTGTGATTTCCAA | 45 | GTGTCGTCTTCCCT | 46 |
| | AGCCAGT | | CCGTTT | |
| Pnp | GGCCCCAACTTTGA | 47 | GGGACTGTGCTCAT | 48 |
| | GACTGT | | GCCAAC | |
| Tdo | ATGCTCAAGGTGAT | 49 | TCCAGAACCGAGAA | 50 |
| | AGCTCGG | | CTGCTG | |
| Qprt | AACTCAACTGCCAA | 51 | CTTGACCTCTGCCA | 52 |
| | GTGTCCT | | CCTTGA | |
| Acmsd | CGAAGGTTTGTGGG | 53 | GCCTTAACACAACG | 54 |
| | TTTGGG | | CTCCATC | |
| Nqo1 | AGCCAATCAGCGTT | 55 | GGCCAGTACAATCA | 56 |
| | CGGTAT | | GGGCTC | |

The results in FIGS. 9C and 9D showed that the expression of several genes involved in the NAD biosynthesis was significantly up-regulated compared to the control when gluconate-CycloZ was administered. These results indicate that gluconate-CycloZ increased the NAD⁺ levels by regulating the expression of genes related to the NAD⁺ synthesis.

### Example 7

Confirmation of Therapeutic Effect with Gluconate-CycloZ Administration in Animal Model of Diabetes and Obesity

The in vivo data of the above examples show the prophylactic effect of gluconate-CycloZ in the KK-Ay mice administered the drug at the beginning of hyperglycemia progression. In clinical settings, patients with T2DM begin drug treatment only when the patients are diagnosed with pre-diabetes or diabetes. Therefore, there is a need to examine the therapeutic effect of gluconate-CycloZ in the later stages of diabetes, which is generally characterized by severe hyperglycemia. According to previous studies, KK-Ay mice showed increased hyperglycemia and insulin resistance with age (Iwatsuka, Hisashi, Akio Shino, and Ziro Suzuoki, “General survey of diabetic features of yellow KK mice,” Endocrinologia japonica 17.1 (1970): 23-35).

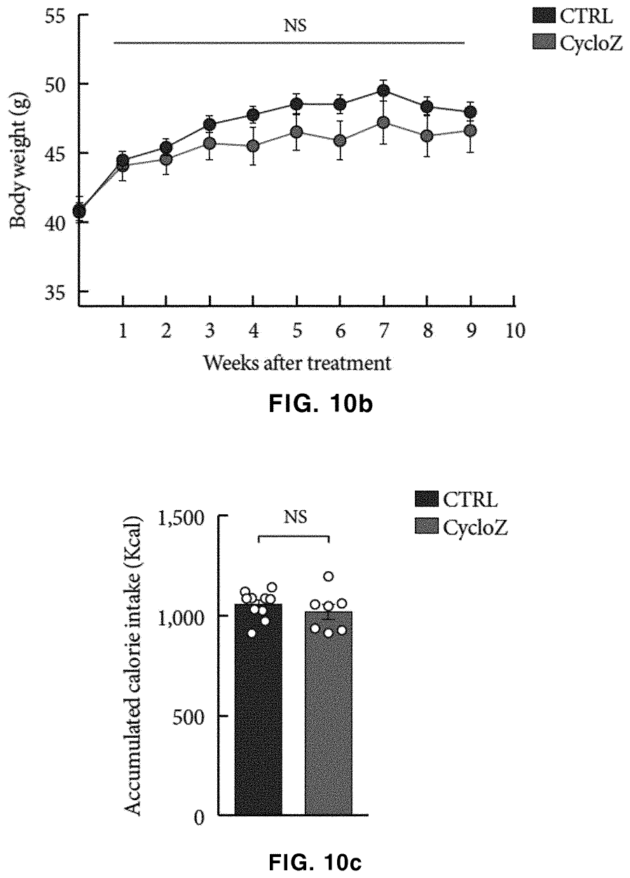

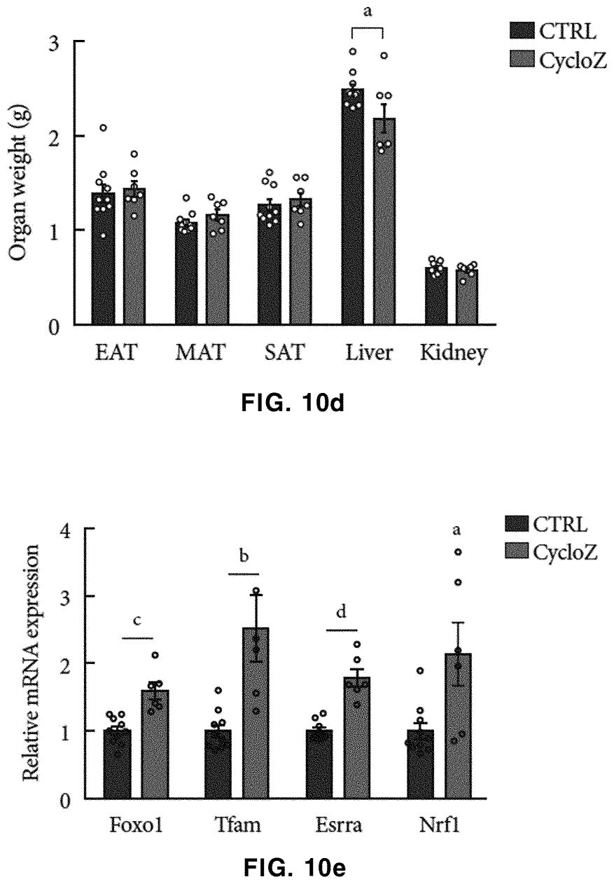

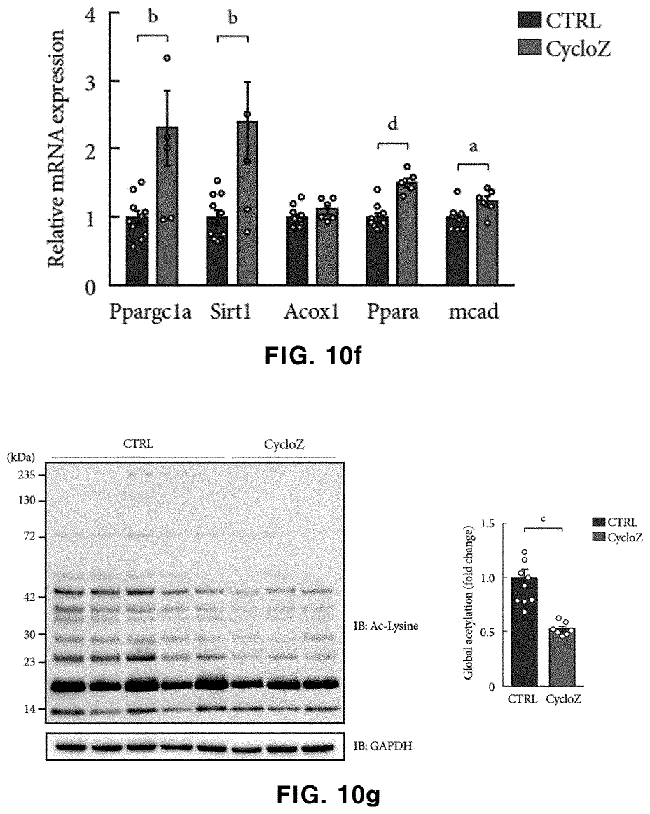

In fact, as a result of measuring the HbA1c levels of KK-Ay mice, it was found that hyperglycemia became more severe at 12 weeks of age compared to 8 weeks of age, as shown in FIG. 10A. Therefore, in this example, gluconate-CycloZ was administered to 12-week-old mice for 8 weeks to investigate the therapeutic effect.

### 7-1. Oral Glucose Tolerance Test and Glycated Hemoglobin Measurement

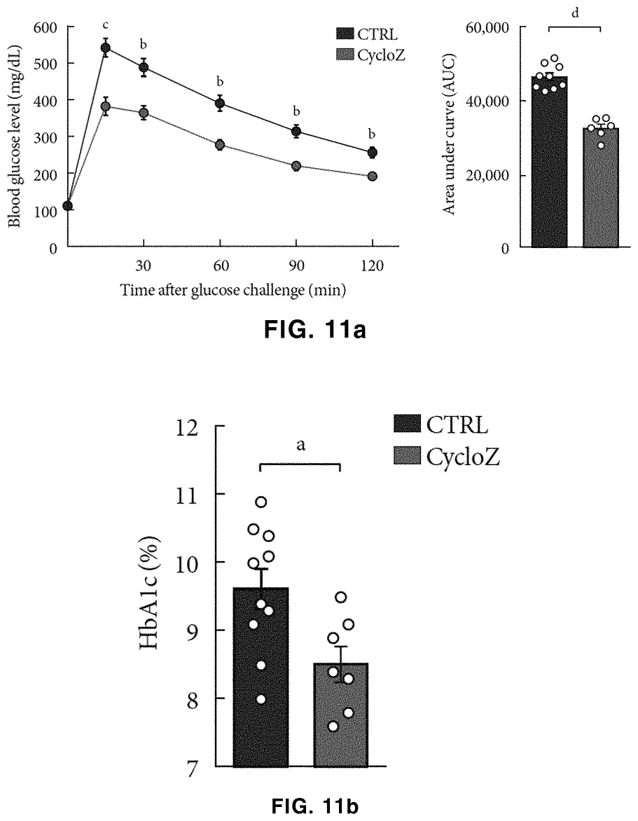

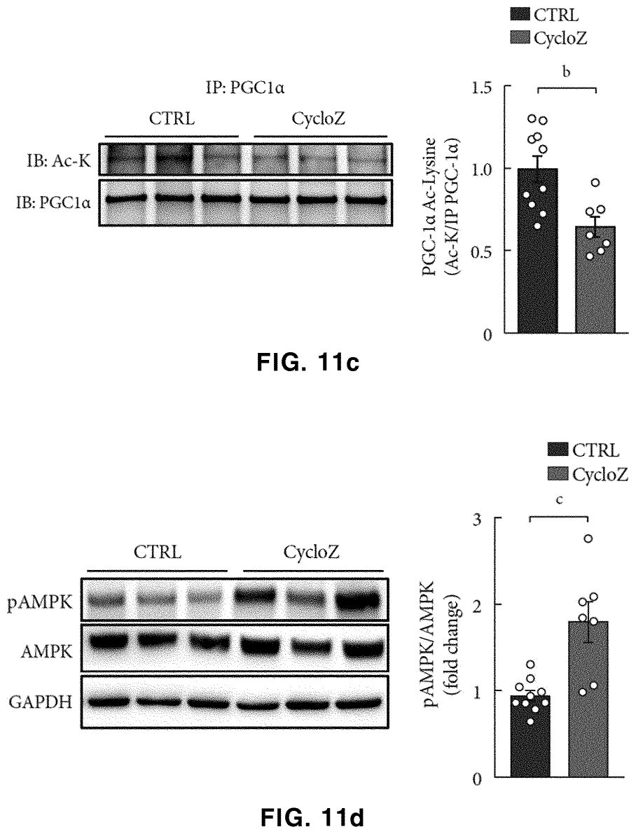

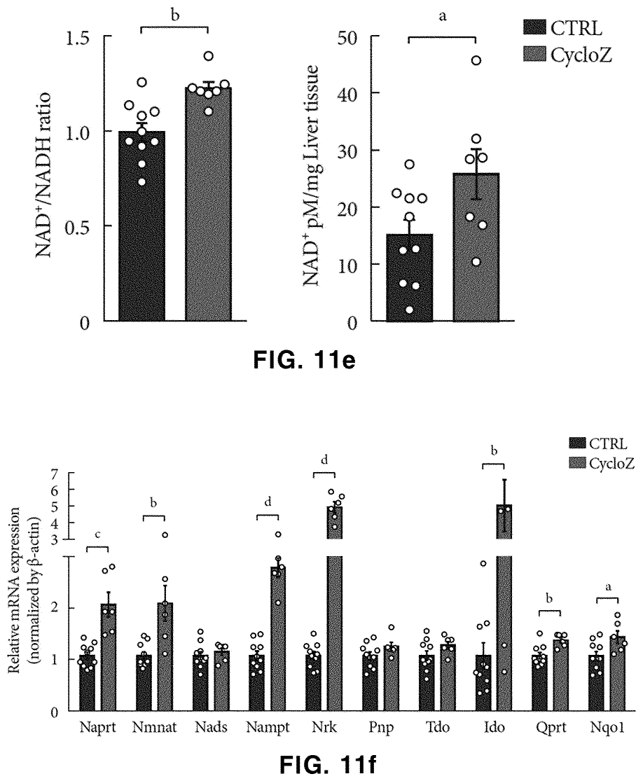

An oral glucose tolerance test and glycated hemoglobin measurement were performed in the same manner as in Example 3-2. As a result, it was confirmed that the glucose tolerance and HbA1c levels were significantly improved by gluconate-CycloZ administration, as shown in FIGS. 11A and 11B.

### 7-2. Western Blot

Western blot was performed in the same manner as in Example 3-7 to examine the acetylation and AMPK phosphorylation of PGC-1α and LKB1. As a result, as shown in FIGS. 11C, 11D, and 10G, it was confirmed that the acetylation of PGC-1α and LKB1 decreased and the AMPK phosphorylation increased, the results of which were observed to be completely consistent with the results of the prevention study.

7-3. mRNA Expression Analysis

After therapeutic administration of CycloZ, RT-qPCR was performed in the same manner as Example 3-5 to investigate the expression of genes related to the mitochondrial biogenesis and function and genes involved in the NAD⁺ synthesis in the liver, and information on the gene-specific primers used is shown in Tables 5 and 6. As seen in the RT-qPCR results of FIGS. 10E and 10F, it was confirmed that the expression of mRNA related to the mitochondrial biogenesis and function increased. Also, as seen in the RT-qPCR results of FIG. 11F, it was confirmed that the expression of genes involved in the NAD⁺ synthesis also increased.

### 7-4. NAD⁺/NADH Quantification

The amount of NAD⁺ and the NAD⁺/NADH ratio upon gluconate-CycloZ administration were quantified in the same manner as in Example 6-1. As a result, as shown in FIG. 11E, it was confirmed that both the amount of NAD⁺ and the NAD⁺/NADH ratio increased. These results indicate that gluconate-CycloZ administration is still effective even in models of more severe diabetes.

### Statistical Analysis

Statistical analysis was performed using Prism software (GraphPad Prism 6, GraphPad Software Inc., San Diego, CA, USA). All data based on the results of Examples 1 to 5 are expressed as mean ±standard error of the mean. Significant differences between two groups were analyzed by the Student's t-test (two-tailed), and multiple comparisons were determined by one-way analysis of variance (ANOVA) followed by a Tukey's post hoc test. P<0.05 was considered statistically significant. A Grubb's test was applied to exclude outlier values.

The composition according to the present invention may be used as a therapeutic agent and a health functional food to prevent, ameliorate, or treat obesity, diabetes, and diabetes accompanied by obesity because the composition is optimized for the type of zinc salt and/or the content ratio of the zinc component to maximize the anti-obesity and anti-diabetic effects when used in combination with CHP.

Although specific parts of the present invention have been described in detail, it will be obvious to a person with ordinary skill in the art that such a specific description is merely a preferred embodiment and the scope of the present invention is not limited thereby. Accordingly, the actual scope of the present invention will be defined by the appended claims and equivalents thereof.

What is claimed is:

1. A method of preventing, ameliorating or treating obesity and diabetes in a subject in need thereof, comprising administering to the subject an effective amount of a composition comprising a zinc gluconate and a cyclo-hispro or a pharmaceutically acceptable salt thereof.

2. The method of claim 1, wherein a zinc cation or zinc element component of the zinc gluconate and the cyclo-hispro or a pharmaceutically acceptable salt thereof are included in a weight ratio of 1 to 5:1 to 4.

3. The method of claim 1, wherein the zinc gluconate and the cyclo-hispro or a pharmaceutically acceptable salt thereof are included in a dose of 15 to 250 mg.

4. The method of claim 3, wherein the zinc gluconate and the cyclo-hispro or a pharmaceutically acceptable salt thereof are included in a weight ratio of 7 to 35:1 to 4.

5. The method of claim 1, wherein the diabetes is type 2 diabetes accompanied by obesity.

6. The method of claim 1, wherein the composition exhibits anti-obesity and anti-diabetic effects through an increase in NAD⁺ synthesis, which regulates Sirt1 deacetylase activity in liver and visceral adipose tissues.

7. A method of increasing β-nicotinamide adenine dinucleotide (NAD⁺) in a subject in need thereof, comprising administering to the subject an effective amount of a composition comprising a zinc gluconate and a cyclo-hispro or a pharmaceutically acceptable salt thereof.

8. The method of claim 7, wherein a zinc cation or zinc element component of the zinc gluconate and the cyclo-hispro or a pharmaceutically acceptable salt thereof are included in a weight ratio of 1 to 5:1 to 4.

9. The method of claim 7, wherein the zinc gluconate and the cyclo-hispro or a pharmaceutically acceptable salt thereof are included in a dose of 15 to 250 mg.

10. The method of claim 7, wherein the zinc gluconate and the cyclo-hispro or a pharmaceutically acceptable salt thereof are included in a weight ratio of 7 to 35:1 to 4.

11. The method of claim 7, wherein the increase in NAD⁺ is an increase in the liver and/or visceral adipose tissue.

12. A method of enhancing Sirt1 deacetylase activity in a subject in need thereof, comprising administering to the subject an effective amount of a composition comprising a zinc gluconate and a cyclo-hispro or a pharmaceutically acceptable salt thereof.

13. The method of claim 12, wherein a zinc cation or zinc element component of the zinc gluconate and the cyclo-hispro or a pharmaceutically acceptable salt thereof are included in a weight ratio of 1 to 5:1 to 4.

14. The method of claim 12, wherein the zinc gluconate and the cyclo-hispro or a pharmaceutically acceptable salt thereof are included in a dose of 15 to 250 mg.

15. The method of claim 12, wherein the zinc gluconate and the cyclo-hispro or a pharmaceutically acceptable salt thereof are included in a weight ratio of 7 to 35:1to 4.
